(19) United States
(12) Patent Application Publication
(10) Pub. No.: US 2020/0354431 A1
(43) Pub. Date: Nov. 12, 2020

(54) MUTABLE COLLAGENOUS TISSUE FROM ECHINODERMS

(71) Applicant: Marine Essence Biosciences Corporation of U.S.A., Chino, CA (US)

(72) Inventors: Danny Qiu, Chino, CA (US); Sergio Madrigal Carballo, Ontario, CA (US)

(21) Appl. No.: 16/405,871

(22) Filed: May 7, 2019

(51) Int. Cl. C07K 14/78; C07K 1/30; A61K 35/616; A61K 8/98; A61K 8/65; A61K 9/00; A61Q 17/04; A61Q 19/08; A61Q 19/00

(52) U.S. Cl. CPC C07K 14/78; C07K 1/30; A61K 35/616; A61K 8/987; A61K 38/00; A61K 9/0014; A61Q 17/04; A61Q 19/08; A61Q 19/007; A61K 8/65

(57) ABSTRACT

The present disclosure relates generally to methods for extracting or purifying collagen type I from marine animals, and uses of the purified collagen type I. Disclosed herein are the methods for the purification of highly pure mutable collagenous tissue from echinoderms. The current disclosure also relates to pharmaceutical, cosmetic and nutritional applications of the mutable collagenous tissue purified using the methods disclosed herein, and to compositions, kits and articles of manufacture made from the mutable collagenous tissue purified using the methods disclosed herein.

### TECHNICAL FIELD

The present technology relates generally to the method for extraction and uses of collagen type I from marine animals. In particular, the present technology relates to the method for the purification of highly pure mutable collagenous tissue from echinoderms, to pharmaceutical, cosmetic and nutritional applications of the mutable collagenous tissue purified using the methods disclosed herein, and to compositions, kits and articles of manufacture made from the mutable collagenous tissue purified using the methods disclosed herein.

### BACKGROUND

The following description of the background of the present technology is provided simply as an aid in understanding the present technology and is not admitted to describe or constitute prior art to the present technology.

Echinoderms are the members of the phylum Echinodermata, which live exclusively in the sea, with no freshwater or terrestrial representatives. Echinoderms fall into five well-defined taxonomic classes: Crinoidea (sea lilies and feather stars); Asteroidea (starfishes); Ophiuroidea (basket stars and brittle stars); Holothuroidea (sea cucumbers); and Echinoidea (sea urchins, sand dollars, and sea biscuits). Adult echinoderms exhibit radial symmetry, typically pentamerous symmetry. Most echinoderms are bottom-dwelling, and live in very shallow water to very deep sea. Dried, powdered bodies and extracts of sea cucumbers have been made into oils, lotions or other topical salves. The genomes of many echinoderms have been sequenced, and transcriptome profiling has also been performed.

### SUMMARY OF THE DISCLOSURE

In one aspect, the present disclosure provides a method for extracting mutable collagenous tissue from echinoderms, the method comprising: treating a connective tissue from an echinoderm with (a) an aqueous solution of an organic solvent; (b) a chelating agent; and (c) an enzyme solution, to thereby obtain a liquid mutable collagenous tissue. In some embodiments, the echinoderms are selected from the group consisting of sea urchins, sea cucumbers, star fish, and a combination thereof. In some embodiments, the organic solvent is selected from the group consisting of ethanol, isopropanol, acetone, ethyl acetate, and a combination thereof. In some embodiments, the aqueous solution of the organic solvent comprises about 25% to about 75% organic solvent. In some embodiments, the treatment time with the organic solvent is in the range of from about 1 minute to about 10 hours. In some embodiments, the chelating agent is selected from the group consisting of ethylenediaminetetraacetic acid (EDTA), ethyleneglycol-bis-(beta-aminoethylether) N,N,N′,N″-tetraacetic acid (EGTA), diethylenetriaminepentaacetic acid (DTPA), and a combination thereof. In some embodiments, the chelating agent further comprises a buffer selected from the group consisting of Tris buffer and phosphate buffer. In some embodiments, the treatment time with the chelating agent is in the range of from about 1 hour to about 10 days.

In some embodiments, the enzyme solution comprises trypsin, bromelain, pepsin, papain, or any combination thereof. In some embodiments, the total enzyme concentration in enzyme solution is at a concentration in the range of from about 0.05% to about 10%. In some embodiments, each enzyme in enzyme solution is used at a concentration in the range of from about 0.05% to about 10%. In some embodiments, the treatment time with the enzyme solution is in the range of from about 12 hours to about 30 days. In some embodiments, the method further comprises: (d) treating the connective tissue with an alkaline solution. In some embodiments, the alkaline solution comprises an alkali metal hydroxide, an alkaline earth metal hydroxide, an alkali metal carbonate, an alkaline earth metal carbonate, an alkali metal bicarbonate, alkaline earth metal bicarbonate or a mixture thereof. In some embodiments, the treatment time with the alkaline solution is in the range of from about 2 hours to about 21 days. In some embodiments, the treatment of the connective tissue with an alkaline solution precedes the treatment of the connective tissue with an enzyme. In some embodiments, the method further comprises: (e) precipitating collagen from the liquid mutable collagenous tissue by adding an inorganic precipitation agent. In some embodiments, the inorganic precipitation agent is an inorganic salt. In some embodiments, the inorganic precipitation agent is sodium chloride, potassium chloride or calcium chloride. In some embodiments, the inorganic precipitation agent is used at a concentration in the range of from about 0.05 M to about 5 M. In some embodiments, the method further comprises separation of the precipitated collagen. In some embodiments, the precipitated collagen is separated by centrifugation at about 1000×g to about 50,000×g. In some embodiments, the method further comprises: (f) dialysis of the precipitated collagen. In some embodiments, the method further comprises: (g) freeze drying the dialyzed collagen to produce a solid mutable collagenous tissue. In some embodiments, the solid mutable collagenous tissue is a powder or a sheet. In some embodiments, the solid mutable collagenous tissue is at least 90%, at least 95%, at least 96%, or at least 97%, at least 98%, at least 99% pure collagen type I. In some embodiments, the solid mutable collagenous tissue is at least 90% pure collagen type I. In some embodiments, the solid mutable collagenous tissue is at least 95% pure collagen type I. In some embodiments, the solid mutable collagenous tissue is at least 98% pure collagen type I.

In one aspect, the present disclosure provides a mutable collagenous tissue obtainable by the method of any of the methods disclosed herein.

In one aspect, the present disclosure provides a pharmaceutical, cosmeceutical or cosmetic composition comprising the mutable collagenous tissue obtainable by any of the methods disclosed herein. In some embodiments, the composition further comprises a pharmaceutically acceptable excipient, additive, preservative, and a mixture thereof. In some embodiments, the composition is formulated to a route of administration selected from the group consisting of oral, sublingual, inhalation, rectal, transmucosal, transdermal, intracavemosal, topical, and injection. In some embodiments, the composition is formulated for topical administration. In some embodiments, the composition is formulated as a lotion, ointment, gel, cream, liquid, emulsion, suppository, drop, spray, hydrogel, dressing, bioadhesive gel, aerosol, paste, foam, sunscreen, or powder. In some embodiments, the composition is attached to a solid substrate. In some embodiments, the composition is formulated as an article or a carrier selected from the group consisting of a bandage, insert, syringe-like applicator, pessary, powder, talc, cleanser, and a patch.

In one aspect, the present disclosure provides a kit comprising a mutable collagenous tissue obtainable by the methods disclosed herein, or any of the compositions disclosed herein. Tn some embodiments, the kit comprises one or more packaging agents, one or more agents for administering the mutable collagenous tissue, one or more cosmetic agents or therapeutic agents, or any combination thereof.

In one aspect, the present disclosure provides a method for treating skin in a subject in need thereof, comprising administering to the subject an effective amount of a cosmetic composition comprising a mutable collagenous tissue obtainable by the methods disclosed herein, or any of the compositions disclosed herein. In some embodiments, the method reduces skin wrinkles, enhances skin moisture content, reduces skin moisture evaporation, inhibits erythema, and suppresses the thickening of the skin epidermis layers, exfoliates the skin, enhances skin elasticity, inhibits erythema, alleviates skin photoaging, firms sagging skin, or any combination thereof. In some embodiments, the cosmetic composition is formulated as a lotion, ointment, gel, cream, liquid, paste, drop, spray, hair restorer, lip cream, patch, thickening gel, water-in-oil emulsion, oil-in-water emulsion, solid, sheet, powder, gel, and mousse. In some embodiments, the cosmetic composition is topically applied.

In one aspect, the present disclosure provides a method for treating a condition in a subject in need thereof, comprising administering to the subject an effective amount of a hydrogel composition comprising a mutable collagenous tissue obtainable by the methods disclosed herein, or any of the compositions disclosed herein. In some embodiments, the condition is selected from the group consisting of (a) a breast imperfections selected from the group consisting of a breast augmentation, a breast reconstruction, and defects due to implant complications; (b) a facial imperfections selected from the group consisting of a facial augmentation, wrinkles, lines, a facial reconstruction, scars, sunken cheeks, hollow cheeks, thin lips, nasal imperfections or defects, retro-orbital imperfections or defects, a facial folds, nasolabial lines, perioral lines, and a marionette line; and (c) a condition requiring an augmentation or a reconstruction of a body part selected from the group consisting of upper arm, lower arm, hand, shoulder, back, neck, torso, abdomen, buttocks, upper leg, lower leg, calves, foot, plantar fat pad, eye, and genitals. In some embodiments, the subject seeks an enlargement, shape change or contour alteration. In some embodiments, the hydrogel composition is administered by intra-epidermal, intra-dermal or subcutaneous injection. In some embodiments, the method promotes cartilage regeneration. In some embodiments, the method is used for the treatment of osteoarthritis.

In one aspect, the present disclosure provides an applicator comprising a mutable collagenous tissue obtainable by the methods disclosed herein, or any of the compositions disclosed herein. In some embodiments, the applicator is a prefilled syringe. In some embodiments, the prefilled syringe comprises a hydrogel composition. In some embodiments, the applicator is selected from the group consisting of a sponge, puff, tube, application nozzle, wipes, spray, a spreadable stick, pencil, and a discharge pad.

In one aspect, the present disclosure provides a nutritional supplement comprising a mutable collagenous tissue obtainable by the methods disclosed herein, or any of the compositions disclosed herein. In some embodiments, the nutritional supplement is formulated as powder, pill, tablet, capsule, beverage, frozen dessert, gelatin dessert, pudding, confectionery, soft gel, chewing gum, candy, gummi candy, gum, caramel, chocolate, tablet sweet, snack, baked good, jelly, jam, yogurt, soup, or stew. In some embodiments, the nutritional supplement further comprises one or more ingredients selected from the group consisting of moisturizers, antioxidants, minerals, metal sequestrants, proteins, enzymes, vitamins, fats, oils, dietary fiber, fruit extracts, vegetable extracts, meat extracts, yeast extract, solvents, buffers, bulking agents, anti-microbial agents, thickeners, antioxidants, surfactants, emulsifiers, solubilizers, solubilizing aids, colorants, perfumes, flavoring agents, sugars, and preservatives. In some embodiments, the nutritional supplement promotes joint health, supports connective tissues, helps maintain a strong immune system, supports restful sleep or promotes healthy digestion. In some embodiments, the nutritional supplement corrects a nutritional insufficiency. In some embodiments, the nutritional supplement stimulates testosterone production to maintain healthy libido, stamina or sexual performance.

### BRIEF DESCRIPTION OF THE DRAWINGS

FIG. 1A (top) illustrates the structure of collagen. The collagen amino acid chains, called α-chains, have a repeating XaaYaaGly amino acid sequence. While Xaa and Yaa can be any amino acids, they are frequently (2S)-proline and (2S,4R)-4-hydroxyproline, respectively. The α-chains are assembled into a left-handed, triple helical structure. Individual triple helices assemble in a complex, hierarchical manner, into collagen fibrils and collagen fibers, as shown. FIG. 1A (bottom) is a photograph illustrating the physical appearance of a purified mutable collagenous tissue (MCT) sheet formed by a dense network of collagen fibers.

FIG. 1B illustrates the structure of mutable collagenous tissue (MCT). As shown, the fibrils are interconnected via proteoglycan/glycosamino glycan (GAG).

FIG. 1C illustrates chemical structure of glycosaminoglycans that crosslink the fibrils of MCT.

FIG. 2 is a flow chart illustrating an exemplary process for extraction of mutable collagenous tissue. A solution of extracted mutable collagenous tissue from sea cucumber and its appearance after freeze drying to produce a white powder are shown.

FIG. 3 shows example sodium dodecyl sulfate-polyacrylamide gel electrophoresis (SDS-PAGE) profiles of the indicated collagen samples. Ladder is a molecular weight ladder used to measure molecular weight based on electrophoretic mobility. Molecular weights of ingredients of the ladder are shown. MC-1 corresponds to the mutable collagenous tissue (MCT) purified from sea cucumber. MC-2 is the purified mutable collagenous tissue (MCT) from sea urchin. MC-3 is purified MCT from starfish. Note the absence of protein bands below 150 kDa in MC-1, MC-2 and MC-3. High molecular weight (>150 kDa), fibrillar collagen triple helix (α1, α2 and β) are shown. Densitometric analysis revealed that MC-1, MC-2 and MC-3 have >98% purity (data not shown). Human: human type I collagen isolated from human fibroblast cells (VitroCol®, Catalog #5007-20ML, Advanced Biomatrix, USA). Rat tail: type I collagen isolated from rat tail (Catalog #C7661-10MG, Sigma Aldrich, USA). Bovine: type-I collagen isolated from bovine skin (PureCol®, Catalog #5005-100ML, Advanced Biomatrix, Aldrich, USA).

FIG. 4A shows a comparison of example gel electrophoresis profiles of MCT collagen isolated from sea urchin (MCT-SU) and sea cucumber (MCT-SC) against different types of native collagen profiles (I, II, III and V). SC=Sea cucumber MCT.

FIG. 4B shows an example analysis of purity of collagen samples as analyzed by SDS-PAGE under non-denaturing conditions. H=human; B=bovine; A. a.=Asterias amurensis; p. l.=Paracentrotus lividus; T a.=Thelenota ananas; C. f.=Cucumaria frondosa.

FIG. 5 shows an example analysis of electrophoretic mobility of collagen samples by SDS-PAGE under denaturing conditions (with a β-mercaptoethanol treatment). MCT=sea cucumber MCT. R=rat collagen type I; C=chicken collagen type I; H=human collagen type I; and B=bovine collagen type I.

FIG. 6A shows example FTIR-ATR spectra for bovine type I collagen, used here as a positive control, and echinoderm mutable collagenous tissue extracted from sea cucumber.

FIG. 6B shows example FTIR-ATR spectra for mutable collagenous tissue (MCT) isolated from sea urchin and sea cucumber.

FIG. 7 shows the amino acid composition of mutable collagenous tissue extracted from sea urchin (MCT-SU) and sea cucumber (MCT-SC), compared to bovine collagen type I isolated from calf skin.

FIG. 8 shows the scanning electron microscopy (SEM) images showing the surface morphology of bovine collagen type I from calf skin, used here as a reference (left image), and mutable collagenous tissue extracted from sea cucumber (right image).

### DETAILED DESCRIPTION

The present disclosure is based, in part, on the discovery that a combination of chemical and enzymatic extracting processes contributes to an increased yield, and higher purity of mutable collagenous tissue. The process described herein also protects the collagen from proteolytic or chemical degradation, and produces mutable collagenous tissue comprising high molecular weight (>150 kDa) collagen. The process described herein also produces mutable collagenous tissue that is free from agents that cause transmissible diseases. Accordingly, the mutable collagenous tissue extracted from echinoderms produced by the methods described herein is useful for a wide variety of applications, including nutritional, cosmetic and therapeutic uses.

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. It is to be understood that this present technology is not limited to particular methods, reagents, compounds compositions or biological systems, which can, of course, vary. The terminology used in the description herein is for the purpose of describing particular embodiments only and is not intended to be limiting.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

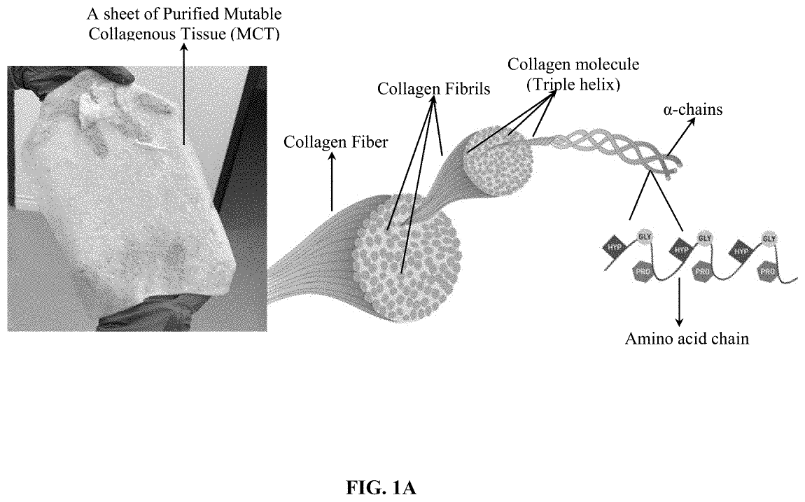

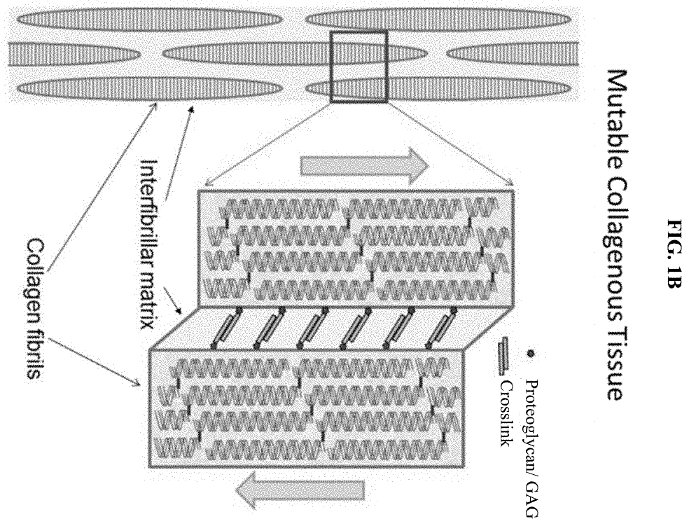

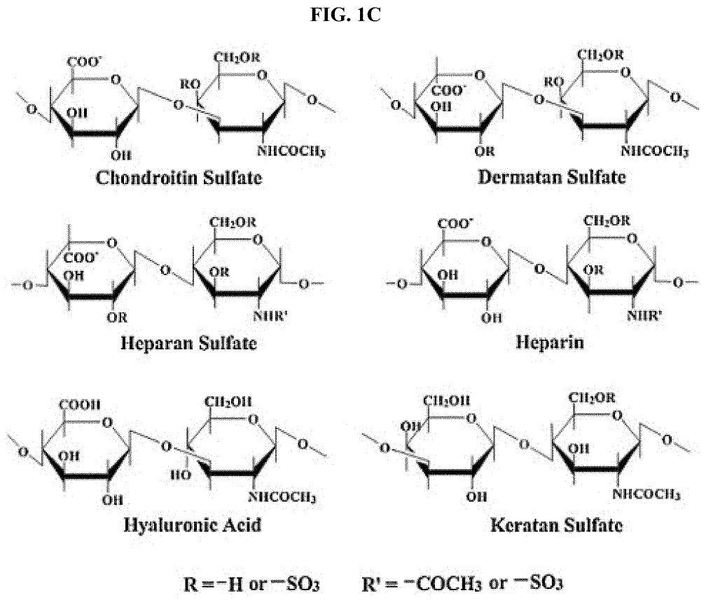

Collagen the most abundant protein in the animal kingdom. It is one of the main structural protein constituents of the extracellular matrix in connective tissues. “Collagen” is a generic term for proteins forming the characteristic triple helix of three polypeptide chains (See, e.g., FIG. 1A), and all members of the collagen family form these supramolecular structures in the extracellular matrix, although their size, function and tissue distribution vary considerably. There are dozens of types of collagens, which are classified into fibril-forming collagens, which include collagen types I, II, III, V, and XI; Fibril-Associated Collagens with Interrupted Triple helices (FACIT collagens), which include collagen types IX, XII, XIV, XVI, XIX, and XX; microfibrillar collagens, which include collagen type VI; short chain collagens, which include collagen types X and VIII; collagens of the basement membrane, which include collagen type IV.

The fibril-forming collagens account for about 90% of the total collagen. Collagen type I, which is the most abundant and best studied collagen, forms more than 90% of the organic mass of bone and is the major collagen of tendons, skin, ligaments, cornea. As shown in FIG. 1A, collagen type I is composed of a triple helix formed of two identical chains (α1), and a third chain (α2), which differs slightly in its chemical composition, which are wound around each other and linked via hydrogen bonds. In animals, individual collagen triple helices assemble in a complex, hierarchical manner that ultimately leads to the macroscopic fibers and networks observed in tissues, such as skin and bone. (See, e. g., FIG. 1A).

Echinoderms are marine invertebrates widespread in all the oceans and are a significant source of food. Echinoderms have a peculiar connective tissue, called mutable collagenous tissue (MCT). The mutable collagenous tissue (MCT) of echinoderms is unique in its ability to “switch” mechanical states rapidly and reversibly. This collagen allows echinoderms to quickly transition from stiff state to soft state and vice versa. For example, sea cucumbers can soften their entire body to allow movements into the tiniest of holes and cracks, yet stiffen again, e.g., to wedge itself into a small crack. MCTs are a unique feature of echinoderms found in each of the five extant taxonomic classes. In contrast, human tissue like skin, tendons and ligaments, cannot switch their mechanical state. Recent studies have shown that a gel-like matrix between the collagen fibrils is the key factor in imparting the echinoderms mutable character. In addition to the functions difference, mutable collagenous tissue differs from, e.g., mammalian collagen with respect to the amino acid primary sequences, associated proteins, and the extent and kind of glycosylation.

As shown in FIG. 1A, MCT comprises discontinuous collagen fibrils organized into collagen fibers. As shown in FIG. 1B, the fibrils form an elastomeric network of fibrillin microfibrils, which are interconnected by a stress-transfer matrix comprising glycosaminoglycan that binds to and aggregates the fibril. As shown in FIG. 1C, the glycosaminoglycans, are long unbranched polysaccharides consisting of a repeating disaccharide unit. The repeating unit consists of an amino sugar (N-acetylglucosamine or N-acetylgalactosamine) along with an uronic sugar (glucuronic acid or iduronic acid) or galactose. Glycosaminoglycans are highly polar and attract water. They are therefore useful to the body as a lubricant or as a shock absorber, being located primarily on the surface of cells or in the extracellular matrix (ECM).

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, immunology, microbiology and recombinant DNA are used. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology. Methods to detect and measure levels of polypeptide gene expression products (i.e., gene translation level) are well-known in the art and include the use of polypeptide detection methods such as antibody detection and quantification techniques. (See also, Strachan & Read, Human Molecular Genetics, Second Edition. (John Wiley and Sons, Inc., NY, 1999)).

### Definitions

As used herein, the singular forms “a,” “an,” and “the” designate both the singular and the plural, unless expressly stated to designate the singular only.

As used herein, the term “about” in reference to a number is generally taken to include numbers that fall within a range of 1%, 5%, or 10% in either direction (greater than or less than) of the number unless otherwise stated or otherwise evident from the context (except where such number would be less than 0% or exceed 100% of a possible value).

As used herein, “and/or” refers to and encompasses any and all possible combinations of one or more of the associated listed items, as well as the lack of combinations when interpreted in the alternative (“or”).

As used herein, a “control” is an alternative sample used in an experiment for comparison purpose. A control can be “positive” or “negative.” For example, where the purpose of the experiment is to determine a correlation of the efficacy of a therapeutic agent for the treatment for a particular type of disease, a positive control (a compound or composition known to exhibit the desired therapeutic effect) and a negative control (a subject or a sample that does not receive the therapy or receives a placebo) are typically employed.

As used herein, an “echinoderm” is a member of the phylum Echinodermata, which includes animals like sea stars, brittle stars, sea urchins, sand dollars, and sea cucumbers.

As used herein, “mutable collagenous tissue,” or “MCT” are used interchangeably herein to mean means the extracellular matrix of connective tissue of echinoderms. “Purified mutable collagenous tissue” is defined as a mutable collagenous tissue that is isolated, or substantially isolated, from other proteins. A purified mutable collagenous tissue may be, e.g., greater than 90% pure, greater than 95% pure, or greater than 98% pure.

As used herein, the term “pharmaceutically acceptable carrier” is intended to include any and all solvents, dispersion media, coatings, antibacterial and antifungal compounds, isotonic and absorption delaying compounds, and the like, compatible with pharmaceutical administration. Pharmaceutically acceptable carriers and their compositions are known to one skilled in the art and are described, for example, in Remington's Pharmaceutical Sciences (20th edition, ed. A. Gennaro, 2000, Lippincott, Williams & Wilkins, Philadelphia, Pa.).

As used herein, the terms “polypeptide”, “peptide” and “protein” are used interchangeably herein to mean a polymer comprising two or more amino acids joined to each other by peptide bonds or modified peptide bonds, i.e., peptide isosteres. Polypeptide refers to both short chains, commonly referred to as peptides, glycopeptides or oligomers, and to longer chains, generally referred to as proteins. Polypeptides may contain amino acids other than the 20 gene-encoded amino acids. Polypeptides include amino acid sequences modified either by natural processes, such as post-translational processing, or by chemical modification techniques that are well known in the art. Such modifications are well described in basic texts and in more detailed monographs, as well as in a voluminous research literature.

As used herein, the term “implant” refers to artificial devices or tissues which are made to replace and act as missing biological structures. These include, for example, dental implants, artificial body parts such as artificial blood vessels or nerve tissues, bone implants, and the like.

As used herein, the term “tissue engineering and regeneration” refers to the engineering and regeneration of new living tissues, such as skin, which includes, e.g., healing diseased, traumatized, unhealthy, or unaesthetic tissue.

As used herein, the terms “pharmaceutical,” “cosmetic,” or “cosmeceutical” composition refers to topical substances that are utilized for pharmaceutical and/or aesthetical purposes. Cosmeceutical or pharmaceutical agents may include substances that exhibit therapeutic activity so as to provide the desired pharmaceutical and/or aesthetical effect. Pharmaceutical, cosmetic or cosmeceutical compositions may be used for firming a defected skin or nail, make ups, gels, lacquers, eye shadows, lip glosses, lipsticks, and the like.

As used herein, the term “medical device” includes, but are not limited to, a hydrogel, bandages, wound dressings, sleeves, films, adhesives and the like.

Methods of Extracting and/or Purifying Mutable Collagenous Tissue from Echinoderms

In one aspect, the present disclosure provides a method for extracting mutable collagenous tissue from echinoderms, the method comprising: (a) treating a connective tissue with an aqueous solution of an organic solvent; and (b) treating the connective tissue with a chelating agent; and (c) treating the connective tissue with an enzyme solution to obtain a liquid mutable collagenous tissue.

Suitable echinoderms for extraction of mutable collagenous tissue include, but are not limited to, sea urchins, sea cucumbers and star fish. Suitable species of echinoderms for extraction of mutable collagenous tissue include, but are not limited to, Strongylocentrotus purpuratus, Arbacia punctulata, Phyllacanthus imperialis, Paracentrotus lividus, Actinopyga echinites, Apostichopus japonicus, Thelenota ananas, Synapta maculata, Holothuria floridana, Holothuria leucospilota, Isostichopus badionotus, Holothuria fuscopunctata, Thelenota rubralineata, Bohadschia argus, Parastichopus californicus, Asterias amurensis, and Cucumaria frondosa.

Suitable organic solvents include, but are not limited to, ethyleneglycol, methanol, ethanol, isopropanol, pyridine, acetonitrile, nitromethane, dimethylsulfoxide, ethylacetate, hexylene glycol, 2,2-thiodiglycol, propylene glycol, dioxane, acetone, dicholoroethane, tetrahydrofuran, dicholoromethane, chloroform, diethylether, benzene, toluene, xylene, carbontetrachloride, cyclohexane, petroleum ether, hexane, pentane, and a combination thereof. In some embodiments, the organic solvent is ethanol, isopropanol, acetone, ethyl acetate or a combination thereof.

The aqueous solution of the organic solvent kills pathogens and inactivates viruses present in the connective tissue. In some embodiments, the aqueous solution of the organic solvent disinfects the connective tissue. In some embodiments, the aqueous solution of the organic solvent sterilizes the connective tissue. In some embodiments, the aqueous solution of the organic solvent comprises a pharmacologically acceptable disinfectant, antiseptic, antibiotic, antimicrobial, antibiotic or the like.

In some embodiments, the aqueous solution of the organic solvent comprises 1%-99% of the organic solvent. In some embodiments, the aqueous solution of the organic solvent comprises about 10%, about 20%, about 30%, about 40%, about 50%, about 60%, about 70%, about 80%, or about 90% organic solvent. In some embodiment, the aqueous solution of the organic solvent comprises organic solvent in the range of from about 5 to about 20%, from about 10 to about 25%, from about 20 to about 50%, from about 25 to about 75%, from about 30 to about 60%, from about 40 to about 70%, or from about 50% to about 80%.

In some embodiments, the connective tissue is treated with the aqueous solution of an organic solvent in the range of from about 1 minute to about 10 hr. In some embodiments, the connective tissue is treated with the aqueous solution of organic solvent in the range of from about 1 minute to about 3 minutes, from about 2 minutes to about 10 minutes, from about 5 minutes to about 15 minutes, from about 10 minutes to about 20 minutes, from about 15 minutes to about 30 minutes, from about 20 minutes to about 1 hour, from about 30 minutes to about 90 minutes, from about 1 hour to about 3 hours, from about 2 hours to about 5 hours, or from about 3 hours to about 10 hours.

In some embodiments, the connective tissue is treated with the aqueous solution of an organic solvent at a temperature in the range of from about −40° C. to about 40° C. In some embodiments, the connective tissue is treated with the aqueous solution of an organic solvent at about −40° C., about −30° C., about −20° C., about −10° C., about −5° C., about 0° C., about 4° C., about 10° C., about 15° C., about 20° C., about 25° C., about 30° C., about 35° C., or about 40° C. In some embodiments, the connective tissue is treated with the aqueous solution of an organic solvent at a temperature in the range of from about −40° C. to about −20° C., from about −30° C. to about −10° C., from about −20° C. to about 0° C., from about −10° C. to about 4° C., from about 0° C. to about 8° C., from about 4° C. to about 10° C., from about 5° C. to about 15° C., from about 10° C. to about 20° C., from about 15° C. to about 25° C., from about 20° C. to about 30° C., from about 25° C. to about 35° C., or from about 30° C. to about 40° C.

In some embodiments, the connective tissue that is treated with the aqueous solution of an organic solvent is recovered by a technique that includes, but not limited to, filtration, centrifugation, and sedimentation. In some embodiments, the connective tissue is further washed with water. In some embodiments, the connective tissue that is treated with the aqueous solution of an organic solvent is subjected to centrifugation with a centrifugal force in the range of from about 1000×g to about 20,000×g, and retrieved from the pellet. In some embodiments, the precipitated collagen is retrieved by centrifugation with a centrifugal force the range of from about 1000×g to about 5,000×g, from about 2500×g to about 7,500×g, from about 5000×g to about 15,000×g, or about 7,500×g to about 20,000×g. In some embodiments, the centrifugation is carried out at temperature the range of from about 0° C. to about 8° C., from about 4° C. to about 10° C., from about 5° C. to about 15° C., from about 10° C. to about 20° C., or from about 15° C. to about 25° C.

Suitable chelating agents include, but are not limited to, ethylenediaminetetraacetic acid (EDTA), ethyleneglycol-bis-(beta-aminoethylether) N,N,N′,N″-tetraacetic acid (EGTA), hydroxyethylenediaminetriacetic acid (HEDTA), nitrilotriaacetic acid (NTA), sodium gluconate, sodium glucoheptonate, diethylenetriaminepentaacetic acid (DTPA), ethylenediaminetetraproprionic acid, triethylenetetraaminehexaacetic acid (TTHA), ethanoldiglycine disodium salt (EDG), diethanolglycine sodium-salt (DEG), and 1,3-propylenediaminetetraacetic acid (PDTA), dicarboxymethyl glutamic acid tetrasodium salt (GLDA), methylglycine-N-N-diacetic acid trisodium salt (MGDA), and iminodisuccinate sodium salt (IDS), and the alkali metal, ammonium and substituted ammonium salts thereof. In some embodiments, the chelating agent is EDTA, EGTA or DTPA.

In some embodiments, the chelating agent is used at a concentration of in the range of from about 0.1 mM to about 500 mM. In some embodiments, the chelating agent is used at a concentration in the range of from about 0.1 mM to about 0.5 mM, from about 0.2 mM to about 1 mM, from about 0.5 mM to about 2.5 mM, from about 1 mM to about 10 mM, from about 2 mM to about 20 mM, from about 5 mM to about 50 mM, from about 25 mM to about 100 mM, from about 50 mM to about 250 mM, or from about 100 mM to about 500 mM.

In some embodiments, the chelating agent is used as an aqueous buffered solution, wherein the buffer is phosphate buffer, phosphate buffered saline, Tris-HCl buffer, Sodium citrate-citric acid buffer, Sodium acetate-acetic acid buffer, Cacodylic acid sodium salt-HCl buffer, MES-NaOH buffer, Sodium dihydrogen phosphate-disodium hydrogen phosphate buffer, Imidazole-HCl buffer, MOPS-KOH buffer, Triethanolamine hydrochloride-NaOH buffer, HEPES-NaOH buffer, Tricine-NaOH buffer, Sodium tetraborate-boric acid buffer, and Bicine-NaOH buffer.

Suitable buffer has a pH between 3-10. In some embodiments, the buffer has a pH in the range of from about 3 to about 5, from about 4 to about 6, from about 5 to about 7, to about 6 to about 8, or about 7 to about 9.

In some embodiments, the buffer is used at a concentration in the range of from about 5 mM to about 1 M. In some embodiments, the chelating agent is used at a concentration of about 5 mM to about 20 mM, from about 10 mM to about 50 mM, from about 25 mM to about 100 mM, from about 50 mM to about 250 mM, from about 100 mM to about 500 mM, or from about 200 mM to about 1 M.

In some embodiments, the connective tissue is treated with the chelating agent for from about 1 hour to about 10 days. In some embodiments, the connective tissue is treated with the chelating agent for a time in the range of from about 1 hour to about 3 hours, from about 2 hours to about 10 hours, from about 5 hours to about 15 hours, from about 10 hours to about 20 hours, from about 12 hours to about 30 hours, from about 18 hours to about 3 days, from about 1 day to about 4 days, from about 2 days to about 5 days, or from about 3 days to about 10 days.

In some embodiments, the connective tissue is treated with the chelating agent at a temperature between about 0° C. to about 40° C. In some embodiments, the connective tissue is treated with the chelating agent at about 0° C., about 4° C., about 10° C., about 15° C., about 20° C., about 25° C., about 30° C., about 35° C., or about 40° C. In some embodiments, the connective tissue is treated with the chelating agent at a temperature in a range of from about 0° C. to about 8° C., from about 4° C. to about 10° C., from about 5° C. to about 15° C., from about 10° C. to about 20° C., from about 15° C. to about 25° C., from about 20° C. to about 30° C., from about 25° C. to about 35° C., or from about 30° C. to about 40° C.

In some embodiments, the connective tissue that is treated with the chelating agent is recovered by a technique that includes, but not limited to, filtration, centrifugation, and sedimentation. In some embodiments, the connective tissue is further washed with water. In some embodiments, the connective tissue that is treated with the chelating agent is subjected to centrifugation with a centrifugal force in the range of from about 1000×g to about 20,000×g, and retrieved from the pellet. In some embodiments, the precipitated collagen is retrieved by centrifugation at about 1000×g to about 5,000×g, about 2500 g to about 10,000×g, or from about 5000 g to about 20,000×g. In some embodiments, the centrifugation is carried out at a temperature in the range of from about 0° C. to about 8° C., from about 4° C. to about 10° C., from about 5° C. to about 15° C., from about 10° C. to about 20° C., or from about 15° C. to about 25° C.

In some embodiments, the treatment of connective tissue with an organic solvent precedes the treatment of the connective tissue with a chelating agent.

Suitable enzymes include, but are not limited to, trypsin, bromelain, pepsin, chymotrypsin, protease VII, thermolysin, subtilisin, ficin, papain microbial proteases, and combinations thereof.

In some embodiments, the enzyme is used at a concentration in the range of from about 0.05% to about 10%. In some embodiments, the enzyme is used at a concentration in the range of from about 0.05% to about 0.25%, from about 0.1% to about 0.5%, from about 0.2% to about 1%, from about 0.5% to about 2.5%, from about 1% to about 5%, or from about 2% to about 10%.

In some embodiments, the connective tissue is treated with the enzyme for a time in the range of from about 12 hours to about 30 days to obtain a liquid mutable collagenous tissue. In some embodiments, the connective tissue is treated with the enzyme for a time in the range of from about 12 hours to about 48 hours, from about 18 hours to about 3 days, from about 24 hours to about 4 days, from about 2 days to about 5 days, from about 3 days to about 10 days, from about 5 days to about 15 days, from about 7 days to about 21 days, or from about 10 days to about 30 days.

In some embodiments, the connective tissue is treated with the enzyme at a temperature between about 0° C. to about 70° C. In some embodiments, the connective tissue is treated with the enzyme at about 0° C., about 4° C., about 10° C., about 15° C., about 20° C., about 25° C., about 30° C., about 35° C., or about 40° C. In some embodiments, the connective tissue is treated with the enzyme at a temperature in the range of from about 0° C. to about 8° C., from about 4° C. to about 10° C., from about 5° C. to about 15° C., from about 10° C. to about 20° C., from about 15° C. to about 25° C., from about 20° C. to about 30° C., from about 25° C. to about 35° C., from about 30° C. to about 40° C., from about 35° C. to about 45° C., from about 40° C. to about 50° C., from about 45° C. to about 55° C., from about 50° C. to about 60° C., from about 55° C. to about 65° C., or from about 60° C. to about 70° C.

In some embodiments, the connective tissue that is treated with the enzyme is subjected to filtration, or centrifugation, and a liquid mutable collagenous tissue is recovered from the filtrate or supernatant. In some embodiments, the connective tissue that is treated with the enzyme is subjected to centrifugation with a centrifugal force in the range of from about 1000×g to about 20,000×g, and the liquid mutable collagenous tissue is retrieved from the supernatant. In some embodiments, the liquid mutable collagenous tissue is retrieved from supernatant following centrifugation with a centrifugal force in the range of from about 1000×g to about 5,000×g, from about 2500×g to about 10,000×g, or from about 5000×g to about 20,000×g. In some embodiments, the centrifugation is carried out at about 0° C. to about 8° C., from about 4° C. to about 10° C., from about 5° C. to about 15° C., from about 10° C. to about 20° C., or from about 15° C. to about 25° C.

In some embodiments, the treatment of connective tissue with a chelating agent precedes the treatment of the connective tissue with an enzyme.

In some embodiments, the method for extracting mutable collagenous tissue from echinoderms further comprises treating the connective tissue with an alkaline solution. Suitable alkaline solution comprises an alkaline chemical, which include, but is not limited to, an alkali metal hydroxide, such as sodium hydroxide or potassium hydroxide; ammonium hydroxide; an alkaline earth metal hydroxide, such as magnesium hydroxide or calcium hydroxide; an alkali metal, or alkaline earth metal carbonate, such as sodium carbonate or potassium carbonate; and an alkali metal or an alkaline earth metal bicarbonate, such as sodium bicarbonate, potassium bicarbonate. Suitable alkaline solution has a pH in the range of from about 8 to about 14. In some embodiments, the suitable alkaline solution has a pH in the range of from about 8 to about 10, from about 9 to about 11, to about 10 to about 12, from about 11 to about 13 or from about 12 to about 14. Suitable alkaline solution has a concentration in the range of from about 0.01 M to about 10 M. In some embodiments, the suitable alkaline solution has a concentration in the range of from about 0.01 M to about 0.1 M, from about 0.05 M to about 0.25 M, from about 0.1 M to about 0.5 M, from about 0.25 M to about 1 M, from about 0.5 M to about 2.5 M, from about 1 M to about 3 M, from about 2 M to about 6 M, or from about 5 M to about 10 M.

In some embodiments, the connective tissue is treated with the alkaline solution for a time in the range of from about 2 hours to about 21 days. In some embodiments, the connective tissue is treated with the alkaline solution for a time in the range of from about 2 hours to about 10 hours, from about 5 hours to about 24 hours, from about 12 hours to about 48 hours, from about 18 hours to about 3 days, from about 24 hours to about 4 days, from about 2 days to about 5 days, from about 3 days to about 10 days, from about 5 days to about 15 days, or from about 7 days to about 21 days.

In some embodiments, the connective tissue is treated with the alkaline solution at a temperature between about 0° C. to about 40° C. In some embodiments, the connective tissue is treated with the alkaline solution at about 0° C., about 4° C., about 10° C., about 15° C., about 20° C., about 25° C., about 30° C., about 35° C., or about 40° C. In some embodiments, the connective tissue is treated with the alkaline solution at a temperature in the range of from about 0° C. to about 8° C., from about 4° C. to about 10° C., from about 5° C. to about 15° C., from about 10° C. to about 20° C., from about 15° C. to about 25° C., from about 20° C. to about 30° C., from about 25° C. to about 35° C., or from about 30° C. to about 40° C.

In some embodiments, the treatment of connective tissue with a chelating agent precedes the treatment of the connective tissue with an alkaline solution, and the treatment of the connective tissue with an alkaline solution precedes the treatment of the connective tissue with an enzyme.

In some embodiments, the method for extracting mutable collagenous tissue from echinoderms further comprises precipitating collagen from the liquid mutable collagenous tissue by adding an inorganic precipitation agent. In some embodiment, the precipitated collagen is the native high molecular weight collagen.

Suitable inorganic precipitation agent is an inorganic salt. In some embodiments, the inorganic precipitation agent is a halide, sulfate, or phosphate salt of an alkali metal or an alkaline earth metal. In some embodiments, the inorganic precipitation agent is sodium chloride, potassium chloride, magnesium chloride, calcium chloride, or a combination thereof. In some embodiments, the inorganic precipitation agent is used at a concentration in the range of from about 0.05 M to about 5 M. In some embodiments, the suitable alkaline solution has a concentration in the range of from about 0.05 M to about 0.25 M, from about 0.1 M to about 0.5 M, from about 0.25 M to about 1 M, from about 0.5 M to about 2.5 M, from about 1 M to about 3 M, or from about 2 M to about 5 M.

In some embodiments, the liquid mutable collagenous tissue is incubated with the inorganic precipitation agent for a time in the range of from about 2 minutes to about 24 hr. In some embodiments, the connective tissue is treated with the alkaline solution for a time in the range of from about 2 minutes to about 10 minutes, from about 5 minutes to about 20 minutes, from about 10 minutes to about 45 minutes, from about 30 minutes to about 2 hours, from about 1 hour to about 3 hours, from about 2 hours to about 5 hours, from about 3 hours to about 10 hours, from about 5 hours to about 15 hours, or from about 8 hours to about 24 hours.

In some embodiments, the liquid mutable collagenous tissue is incubated with the inorganic precipitation agent at a temperature between about 0° C. to about 40° C. In some embodiments, the liquid mutable collagenous tissue is incubated with the inorganic precipitation agent at a temperature in the range of about 0° C., about 4° C., from about 10° C., from about 15° C., from about 20° C., from about 25° C., from about 30° C., from about 35° C., or about 40° C. In some embodiments, the liquid mutable collagenous tissue is incubated with the inorganic precipitation agent at a temperature between about 0° C. to about 8° C., from about 4° C. to about 10° C., from about 5° C. to about 15° C., from about 10° C. to about 20° C., from about 15° C. to about 25° C., from about 20° C. to about 30° C., from about 25° C. to about 35° C., or from about 30° C. to about 40° C.

In some embodiment, the precipitated collagen is separated from supernatant by centrifugation and recovery of the pellet. In some embodiments, the precipitated collagen is separated by centrifugation at a centrifugal force in the range of from about 1000×g to about 50,000×g, and retrieved from the pellet. In some embodiments, the precipitated collagen is retrieved by centrifugation at a centrifugal force in the range of from about 1000×g to about 5,000×g, from about 2500×g to about 10,000×g, from about 5000^{x} g to about 20,000×g, or from about 10,000×g to about 50,000×g. In some embodiments, the centrifugation is carried out at a temperature in the range of from about 0° C. to about 8° C., from about 4° C. to about 10° C., from about 5° C. to about 15° C., from about 10° C. to about 20° C., or from about 15° C. to about 25° C.

In some embodiments, the pellet is resuspended in an acidic solution. In some embodiments, the acidic solution comprises an acid, which includes, but is not limited to acetic acid, citric acid, propionic acid, lactic acid, and hydrochloric acid. In some embodiments, the acidic solution comprises an acid in the concentration range of from about 0.01 M to about 5 M. The acidic solution comprises an acid in the concentration range of from about 0.01 M to about 0.1 M, from about 0.05 M to about 0.25 M, from about 0.1 M to about 0.5 M, from about 0.25 M to about 1 M, from about 0.5 M to about 2.5 M, from about 1 M to about 3 M, or from about 2 M to about 5 M. In some embodiment, the acidic solution has a pH in the range of from about 1 to about 3, from about 2 to about 4, from about 3 to about 5, or from about 4 to about 6. In some embodiments, the acidic solution is ice cold. In some embodiments, the temperature of the acidic solution has is about 0° C., about 4° C., about 10° C., about 15° C., about 20° C., or about 25° C.

In some embodiments, the treatment of connective tissue with an enzyme precedes precipitating the native high molecular weight collagen from the liquid mutable collagenous tissue by adding an inorganic precipitation agent.

In some embodiments, the method for extracting mutable collagenous tissue from echinoderms further comprises dialysis of the pellet, which is resuspended in an acidic solution. In some embodiments, the pellet is dialyzed against water or an aqueous buffer. In some embodiments, the buffer is phosphate buffer, phosphate buffered saline, Tris-HCl buffer, Sodium citrate-citric acid buffer, Sodium acetate-acetic acid buffer, Cacodylic acid sodium salt-HCl buffer, MES-NaOH buffer, Sodium dihydrogen phosphate-disodium hydrogen phosphate buffer, Imidazole-HCl buffer, MOPS-KOH buffer, Triethanolamine hydrochloride-NaOH buffer, HEPES-NaOH buffer, Tricine-NaOH buffer, Sodium tetraborate-boric acid buffer, and Bicine-NaOH buffer. In some embodiments, the buffer has a pH between 3-9. In some embodiments, the buffer has a pH in the range of from about 3 to about 5, from about 4 to about 6, from about 5 to about 7, to about 6 to about 8, or from about 7 to about 9. In some embodiments, the buffer is used at a concentration in the range of from about 5 mM to about 250 mM. In some embodiments, the buffer has a concentration in the range of from about 5 mM to about 50 mM, from about 25 mM to about 100 mM, or from about 50 mM to about 250 mM.

In some embodiments, the dialysis is carried out at a temperature between about 0° C. to about 40° C. In some embodiments, the dialysis is carried out at about 0° C., about 4° C., about 10° C., about 15° C., about 20° C., about 25° C., about 30° C., about 35° C., or about 40° C. In some embodiments, the dialysis is carried out at a temperature in a range of from about 0° C. to about 8° C., from about 4° C. to about 10° C., from about 5° C. to about 15° C., from about 10° C. to about 20° C., from about 15° C. to about 25° C., from about 20° C. to about 30° C., from about 25° C. to about 35° C., or from about 30° C. to about 40° C.

In some embodiments, the method for extracting mutable collagenous tissue from echinoderms further comprises freeze drying to produce solid mutable collagenous tissue.

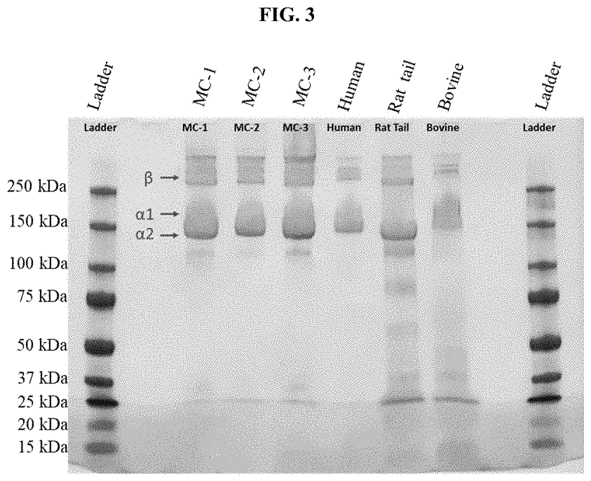

The mutable collagenous tissue obtained by this process is highly pure (See FIG. 3). In some embodiments, the solid mutable collagenous tissue is a sheet composed of collagen type I. In some embodiments, the solid mutable collagenous tissue is a powder composed of collagen type I. In some embodiments, mutable collagenous tissue obtained by the method disclosed here is at least 90% pure collagen type I. In some embodiments, mutable collagenous tissue obtained by the method disclosed here is at least 92% pure collagen type I. In some embodiments, mutable collagenous tissue obtained by the method disclosed here is at least 95% pure collagen type I. In some embodiments, mutable collagenous tissue obtained by the method disclosed here is at least 98% pure collagen type I.

In some embodiments, the method for extracting mutable collagenous tissue from echinoderms comprises the following steps in the following order:
a. Treating a connective tissue with an aqueous solution of an organic solvent;
b. Treating the connective tissue with a chelating agent; and
c. Treating the connective tissue with an enzyme to yield a liquid mutable collagenous tissue.

In some embodiments, the method for extracting mutable collagenous tissue from echinoderms comprises the following steps in the following order:
a. Treating a connective tissue with an aqueous solution of an organic solvent;
b. Treating the connective tissue with a chelating agent;
c. Treating the connective tissue with an alkaline solution; and
d. Treating the connective tissue with an enzyme to yield a liquid mutable collagenous tissue.

In some embodiments, the method for extracting mutable collagenous tissue from echinoderms comprises the following steps in the following order:
a. Treating a connective tissue with an aqueous solution of an organic solvent;
b. Treating the connective tissue with a chelating agent;
c. Treating the connective tissue with an enzyme to yield a liquid mutable collagenous tissue; and
d. Dialyzing the mutable collagenous tissue against deionized water to obtain a native mutable collagenous tissue solution.

In some embodiments, the method for extracting mutable collagenous tissue from echinoderms comprises the following steps in the following order:
a. Treating a connective tissue with an aqueous solution of an organic solvent;
b. Treating the connective tissue with a chelating agent;
c. Treating the connective tissue with an enzyme to yield a liquid mutable collagenous tissue;
d. Dialyzing the mutable collagenous tissue against deionized water to obtain a native mutable collagenous tissue solution; and
e. Freeze drying the native collagen solution to obtain native mutable collagenous tissue sheet or powder.

In some embodiments, the method for extracting mutable collagenous tissue from echinoderms comprises the following steps in the following order:
a. Treating a connective tissue with an aqueous solution of an organic solvent;
b. Treating the connective tissue with a chelating agent;
c. Treating the connective tissue with an alkaline solution;
d. Treating the connective tissue with an enzyme to yield a liquid mutable collagenous tissue;
e. Precipitating collagen from the liquid mutable collagenous tissue by adding an inorganic precipitation agent;
f. Dialyzing the mutable collagenous tissue against deionized water to obtain a native mutable collagenous tissue solution;
g. Freeze drying the native collagen solution to obtain native mutable collagenous tissue sheet or powder.

In some embodiments, treating a connective tissue with an aqueous solution of the organic solvent comprises (i) collecting the echinoderm specimens, and dissecting them, (ii) isolating connective tissue from the body wall, (iii) treating a connective tissue with an aqueous solution of an organic solvent (comprising ethanol, isopropanol, acetone and/or ethyl acetate, in concentrations ranging 30 to 50%) for 10 to 60 minutes, (iv) centrifugation at about 1000 to about 20,000×g for about 10 min to 60 min, (v) collecting the connective tissue, and (vi) and washing with in deionized water.

In some embodiments, treating a connective tissue with a chelating agent comprises (i) treating a connective tissue with an aqueous solution of a chelating agent (comprising ethylene glycol tetraacetic acid (EGTA), ethylene diamine tetraacetic acid (EDTA) and/or diethylene triamine pentaacetic acid (DTPA), together in phosphate buffer saline (PBS), tris buffer or acetate buffer) for 12 to 24 hours, (ii) centrifugation at about 1000 to about 20,000×g for about 10 min to 60 min, (iii) collecting the connective tissue, and (iv) and washing with in deionized water.

In some embodiments, treating a connective tissue with an alkaline solution, which comprises (i) treating a connective tissue with an alkaline solution (comprising 0.1 M to 1.0 M sodium hydroxide and/or sodium bicarbonate) for 24 to 48 hours, (ii) centrifugation at about 1000 to about 20,000×g for about 10 min to 60 min, iii) collecting the connective tissue, and (iv) and washing with in deionized water.

In some embodiments, treating a connective tissue with an enzyme to yield a liquid mutable collagenous tissue comprises (i) treating a connective tissue with an enzyme (comprising 0.5 to 1.0% trypsin, bromelain, pepsin or papain in a suitable buffer) for 48 to 72 hours, (ii) centrifugation at about 1000 to about 20,000×g for about 10 min to 60 min, (iii) collecting the supernatant, which is the liquid mutable collagenous tissue.

In some embodiments, precipitating collagen from the liquid mutable collagenous tissue by adding an inorganic precipitation agent comprises (i) adding sodium chloride, potassium chloride and/or calcium chloride to a concentration of 0.5 to 1.0 M to the liquid mutable collagenous tissue, (ii) centrifugation at about 1000 to about 20,000×g for about 10 min to 60 min, iii) collecting the pellet, and (iv) and resuspending the pellet in 0.1 to 0.5 M acetic acid, citric acid, propionic acid and/or hydrochloric acid to obtain a resuspended pellet.

In some embodiments, the method for extracting mutable collagenous tissue from echinoderms comprises the following steps in the following order:
a. Treating a connective tissue with an aqueous solution of an organic solvent, which comprises (i) collecting the echinoderm specimens, and dissecting them, (ii) isolating connective tissue from the body wall, (iii) treating a connective tissue with an aqueous solution of an organic solvent (comprising ethanol, isopropanol, acetone and/or ethyl acetate, in concentrations ranging 30 to 50%) for 10 to 60 minutes, (iv) centrifugation at about 1000 to about 20,000×g for about 10 min to 60 min, (v) collecting the connective tissue, and (vi) and washing with in deionized water;
b. Treating the connective tissue with a chelating agent, which comprises (i) treating a connective tissue with an aqueous solution of a chelating agent (comprising ethylene glycol tetraacetic acid (EGTA), ethylene diamine tetraacetic acid (EDTA) and/or diethylene triamine pentaacetic acid (DTPA), together in phosphate buffer saline (PBS), tris buffer or acetate buffer) for 12 to 24 hours, (ii) centrifugation at about 1000 to about 20,000×g for about 10 min to 60 min, (iii) collecting the connective tissue, and (iv) and washing with in deionized water;
c. Treating the connective tissue with an alkaline solution, which comprises (i) treating a connective tissue with an alkaline solution (comprising 0.1 M to 1.0 M sodium hydroxide and/or sodium bicarbonate) for 24 to 48 hours, (ii) centrifugation at about 1000 to about 20,000×g for about 10 min to 60 min, iii) collecting the connective tissue, and (iv) and washing with in deionized water;
d. Treating the connective tissue with an enzyme to yield a liquid mutable collagenous tissue, which comprises (i) treating a connective tissue with an enzyme (comprising 0.5 to 1.0% trypsin, bromelain, pepsin or papain in a suitable buffer) for 48 to 72 hours, (ii) centrifugation at about 1000 to about 20,000×g for about 10 min to 60 min, (iii) collecting the supernatant, which is the liquid mutable collagenous tissue;
e. Precipitating collagen from the liquid mutable collagenous tissue by adding an inorganic precipitation agent, which comprises (i) adding sodium chloride, potassium chloride and/or calcium chloride to a concentration of 0.5 to 1.0 M, (ii) centrifugation at about 1000 to about 20,000×g for about 10 min to 60 min, iii) collecting the pellet, and (iv) and resuspending the pellet in 0.1 to 0.5 M acetic acid, citric acid, propionic acid and/or hydrochloric acid to obtain a resuspended pellet;
f. Dialyzing the resuspended pellet against deionized water for 72 hours using cellulose membranes with 10 kDa molecular weight cut-off to obtain a native mutable collagenous tissue solution;
g. Freeze drying the native collagen solution to obtain native mutable collagenous tissue powder.

In some embodiments, the method for extracting mutable collagenous tissue from echinoderms comprises the steps of: 1) soaking isolated connective tissue in organic extracting solution for disinfection and extraction of fat-related compounds; 2) pre-treating the connective tissue extract with a chelating buffer solution to eliminate heavy metals and related impurities; 3) soaking the connective tissue extract alkaline solution to eliminate non-collagenous tissue; 4) soaking the connective tissue extract in an enzyme to yield to a liquid mutable collagenous tissue; 5) extracting the native high molecular weight collagen from liquid mutable collagenous tissue isolate by adding inorganic precipitation agent; 6) dissolving of the isolated native collagen in collagen dissolving solution, and 7) isolating high-purity native collagen powder by dialysis followed by freeze drying.

In some embodiments, the connective tissue is excised from echinoderm species including, but not limited to, sea urchins, sea cucumbers and/or star fish. Species of echinoderms for extraction of mutable collagenous tissue could be selected from the group composed by, but not limited to, Strongylocentrotus purpuratus, Paracentrotus lividus, Apostichopus japonicus, Thelenota ananas, Parastichopus californicus, Asterias amurensis, Cucumaria frondosa.

In some embodiments, the organic extracting solution is composed by aqueous solutions of ethanol, isopropanol, acetone or ethyl acetate, in concentrations ranging 30 to 50% and total reaction time between 10 to 60 minutes.

In some embodiments, the chelating buffer solution can be composed by a chelating agent from the group consisting of ethylene glycol tetraacetic acid (EGTA), ethylene diamine tetraacetic acid (EDTA) or diethylene triamine pentaacetic acid (DTPA), together with a buffer solution composed by phosphate buffer saline (PBS), tris buffer or acetate buffer, in ratios ranging 1:5 to 1:10, respectively, and total reaction time between 12 to 24 hours.

In some embodiments, the alkaline solution is composed of a caustic aqueous solution comprising sodium hydroxide or sodium bicarbonate at a concentration ranging from 0.1 to 1.0 M and total reaction time between 24 to 48 hours.

In some embodiments, the enzyme, includes but not limited to, trypsin, bromelain, pepsin or papain at concentrations ranging from 0.5 to 1.0% and total reaction time between 48 to 72 hours.

In some embodiments, the inorganic precipitation agent is an inorganic salt, including, but not limited to, sodium chloride, potassium chloride or calcium chloride, at concentrations ranging from 0.5 to 1.0 M.

In some embodiments, collagen dissolving solution is composed by an acid including, but not limited to, acetic acid, citric acid, propionic acid or hydrochloric acid, in concentrations ranging 0.1 to 0.5 M.

In some embodiments, the mutable collagenous tissue purified by the methods disclosed herein shows a macromolecular structure of collagen type I.

In some embodiments, the mutable collagenous tissue purified by the methods disclosed herein shows a macromolecular structure of collagen type I, exhibits high purity (>98%), showing no degradation of the main collagen structure (chemical or biological) or the presence of contaminants from extraction processes (small proteins or peptides).

In some embodiments, the mutable collagenous tissue purified by the methods disclosed herein shows high molecular weight (>150 kDa),

In some embodiments, the mutable collagenous tissue purified by the methods disclosed herein shows high molecular weight (>150 kDa), as shown by SDS gel electrophoresis.

In some embodiments, the mutable collagenous tissue purified by the methods disclosed herein exhibits α1, and α2 chains, as shown by SDS-PAGE electrophoresis. In some embodiments, the mutable collagenous tissue purified by the methods disclosed herein exhibits protein bands in α, β and γ regions, as shown by SDS-PAGE electrophoresis. In some embodiments, the mutable collagenous tissue purified by the methods disclosed herein exhibits protein bands at or above 150 kDa, as shown by SDS-PAGE electrophoresis. In some embodiments, the mutable collagenous tissue purified by the methods disclosed herein exhibits less than 2% impurities and degradation products that run substantially below 150 kDa, as shown by SDS-PAGE electrophoresis.

In one aspect, the present disclosure provides a mutable collagenous tissue obtainable by any of the methods described above. In some embodiments, the mutable collagenous tissue obtainable by any of the methods described herein is a liquid mutable collagenous tissue, a sheet of mutable collagenous tissue, a mutable collagenous tissue powder or a combination thereof.

### Compositions

In one aspect, the present disclosure provides a pharmaceutical, cosmeceutical or cosmetic composition comprising the mutable collagenous tissue obtainable by any of the methods described herein. In some embodiments, the provides a pharmaceutical, cosmeceutical or cosmetic composition comprises from about 0.01% to about 99.9% by weight of the composition mutable collagenous tissue obtainable by any of the methods described herein. In some embodiments, the mutable collagenous tissue obtainable is mixed with one or more pharmaceutically acceptable excipients, additives, and/or preservatives. In some embodiments, the excipients include diluents, lubricants, binders, disintegrates, buffers, dispersants, surfactants, coloring agents, perfumes, aromatics, or sweeteners.

Suitable pharmaceutically acceptable excipients or pharmaceutically acceptable carriers, may include solvents, dispersion media, coatings, isotonic and absorption delaying agents and the like, and combinations comprising one or more of the foregoing carriers as described, for instance, in Remington's Pharmaceutical Sciences, 15th Ed. Easton: Mack Publishing Co. pp. 1405-1412 and 1461-1487 (1975), and The National Formulary XIV 14th Ed., Washington: American Pharmaceutical Association (1975). Suitable carriers include, but are not limited to, calcium carbonate, carboxymethylcellulose, cellulose, citric acid, dextrate, dextrose, ethyl alcohol, glucose, hydroxymethylcellulose, lactose, magnesium stearate, maltodextrin, mannitol, microcrystalline cellulose, oleate, polyethylene glycols, potassium diphosphate, potassium phosphate, saccharose, sodium diphosphate, sodium phosphate, sorbitol, starch, stearic acid and its salts, sucrose, talc, vegetable oils, water, and combinations comprising one or more of the foregoing carriers. Except insofar as any conventional media or agent is incompatible with the emulsions of the present invention, their use in pharmaceutical, cosmeceutical or cosmetic compositions is contemplated. Supplementary active ingredients also can be incorporated into the pharmaceutical, cosmeceutical or cosmetic compositions.

The pharmaceutical, cosmeceutical or cosmetic compositions comprising the mutable collagenous tissue disclosed herein described herein can be formulated for various routes of administration. Suitable routes of administration include, but are not limited to, oral, sublingual, inhalation, rectal, transmucosal, transdermal, intracavemosal, topical, intestinal or parenteral delivery, including intramuscular, subcutaneous and intramedullary injections as well as intrathecal, direct intraventricular, intravenous, intraperitoneal, intranasal, or intraocular injections.

The pharmaceutical, cosmeceutical or cosmetic compositions for topical administration include, but are not limited to, lotions, ointments, gels, creams, liquids, emulsions, suppositories, drops, sprays, hydrogel, bioadhesive gels, aerosols, pastes, foams, sunscreens, and powders, or in the form of an article or carrier, such as a bandage, insert, syringe-like applicator, pessary, powder, talc or other solid, cleanser (leave on and wash off product), and agents that favor penetration within the pilosebaceous gland.

The pharmaceutical, cosmeceutical or cosmetic compositions for topical administration may additionally comprise organic solvents, emulsifiers, gelling agents, moisturizers, stabilizers, surfactants, wetting agents, preservatives, time release agents, and minor amounts of humectants, sequestering agents, dyes, perfumes, and other components commonly used in cosmetic or pharmaceutical compositions for topical and mucosal administration.

In some embodiments, the pharmaceutical, cosmeceutical or cosmetic compositions may comprise a penetration-enhancing agent for enhancing penetration of the nanoemulsion through the stratum corneum and into the epidermis or dermis. Suitable penetration-enhancing agents include, but are not limited to, alcohols such as ethanol, triglycerides, fatty acids (e.g., lauric acid, oleic acid, myristic acid), fatty alcohols (e.g., myristyl or oleoyl alcohols), fatty esters (e.g., isopropyl myristate), lipids, anionic and nonionic surfactants and aloe compositions. The amount of the penetration-enhancing agent may comprise from about 0.5% to about 40% by weight of the composition.

Topical administration includes administration to the skin, mucosa, and squamous epithelium, including surface of the hair follicle and pilosebaceous unit. In some embodiments, the composition enters the epidermis, dermis, mucosa, squamous epithelium, or any combination thereof. In some embodiments, the composition permeates into the epidermis and dermis via the follicular route using skin pores and hair follicles. In some embodiments, the composition diffuses through the skin, skin pores, nail, scalp, hair follicles, lateral or proximal folds, nail, hyponichium, or a combination thereof.

In some embodiments, the composition for delivery via a “patch” comprising the mutable collagenous tissue is envisioned. As used herein a “patch” comprises at least a topical composition and a covering layer, such that the patch can be placed over the area to be treated. Preferably, the patch is designed to maximize delivery through the stratum corneum and into the epidermis or dermis, while minimizing absorption into the circulatory system, and little to no skin irritation, reducing lag time, promoting uniform absorption, and reducing mechanical rub-off and dehydration.

Adhesives for use with the drug-in-adhesive type patches are well known in the art. Suitable adhesive include, but are not limited to, polyisobutylenes, silicones, and acrylics. These adhesives can function under a wide range of conditions, such as, high and low humidity, bathing, sweating etc. Preferably the adhesive is a composition based on natural or synthetic rubber; a polyacrylate such as, polybutylacrylate, polymethylacrylate, poly-2-ethylhexyl acrylate; polyvinylacetate; polydimethylsiloxane; or and hydrogels (e.g., high molecular weight polyvinylpyrrolidone and oligomeric polyethylene oxide). The most preferred adhesive is a pressure sensitive acrylic adhesive, for example Durotak® adhesives (e.g., Durotak® 2052, National Starch and Chemicals). The adhesive may comprise a thickener, such as a silica thickener (e.g., Aerosil, Degussa, Ridgefield Park, N.J.) or a crosslinker such as aluminumacetylacetonate.

Suitable release liners include but are not limited to occlusive, opaque, or clear polyester films with a thin coating of pressure sensitive release liner (e.g., silicone-fluorsilicone, and perfluorcarbon based polymers.

Backing films may be occlusive or permeable and are derived from synthetic polymers like polyolefin oils polyester, polyethylene, polyvinylidine chloride, and polyurethane or from natural materials like cotton, wool, etc. Occlusive backing films, such as synthetic polyesters, result in hydration of the outer layers of the stratum corneum while non-occlusive backings allow the area to breath (i.e., promote water vapor transmission from the skin surface). More preferably the backing film is an occlusive polyolefin foil (Alevo, Dreieich, Germany). The polyolefin foil is preferably about 0.6 to about 1 mm thick.

The shape of the patch can be flat or three-dimensional, round, oval, square, and have concave or convex outer shapes, or the patch or bandage can also be segmented by the user into corresponding shapes with or without additional auxiliary means.

The pharmaceutical, cosmeceutical or cosmetic compositions may be applied in a single administration or in multiple administrations. The pharmaceutical, cosmeceutical or cosmetic compositions can be applied for any suitable time period, such as once or multiples times per day. The compositions can be applied for at least once a week, at least twice a week, at least once a day, at least twice a day, multiple times daily, multiple times weekly, biweekly, at least once a month, or any combination thereof. The pharmaceutical, cosmeceutical or cosmetic compositions are applied for a period of time of about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years. Between applications, the application area may be washed to remove any residual mutable collagenous tissue.

Preferably, the pharmaceutical, cosmeceutical or cosmetic compositions are applied to the skin area in an amount of from about 0.001 mL/cm² to about 5.0 mL/cm². An exemplary application amount and area is about 0.2 mL/cm², although any amount from 0.001 mL/cm² up to about 5.0 mL/cm² can be applied. Following topical administration, the nanoemulsion may be occluded or semi-occluded. Occlusion or semi-occlusion may be performed by overlaying a bandage, polyoleofin film, impermeable barrier, or semi-impermeable barrier to the topical preparation. Preferably, after application, the treated area is covered with a dressing.

In some embodiments, the pharmaceutical, cosmeceutical or cosmetic compositions described herein are formulated for mucosal delivery, for example by contacting any one of the compositions described herein to a nasal mucosal epithelium, a bronchial or pulmonary mucosal epithelium, an oral, gastric, intestinal or rectal mucosal epithelium, or a vaginal mucosal epithelium. In some embodiments, the compositions described herein are formulated for intranasal delivery, (e.g., nasal mucosal delivery or intranasal mucosal delivery).

The pharmaceutical, cosmeceutical or cosmetic compositions for oral administration include, but are not limited to, tablets, pills, soft or hard capsules, granules, powders, solution, or emulsion.

In some embodiments pharmaceutical, cosmeceutical or cosmetic compositions for topical administration include a hydrogel, an implant, an artificial body part, a tissue engineering and regeneration system, and a wound dressing, or a medical device.

The compositions comprising the mutable collagenous tissue disclosed herein can include a carrier, which can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thiomerasol, and the like. In many cases, it will be advantageous to include isotonic agents, for example, sugars, polyalcohols such as mannitol, sorbitol, or sodium chloride in the composition.

The mutable collagenous tissue disclosed herein can be formulated in a carrier system. The carrier can be a colloidal system. The carrier can also be a polymer, e.g., a biodegradable, biocompatible polymer matrix. In one embodiment, one or more therapeutic agent can be embedded in the polymer matrix, while maintaining the agent's structural integrity. The polymer may be natural, such as polypeptides, proteins or polysaccharides, or synthetic, such as poly α-hydroxy acids. Examples include carriers made of, e.g., fibronectin, elastin, cellulose acetate, cellulose nitrate, polysaccharides, fibrin, and combinations thereof. In one embodiment, the polymer is poly-lactic acid (PLA) or copoly lactic/glycolic acid (PGLA). The polymeric matrices can be prepared and isolated in a variety of forms and sizes, including liposomes, nanofibers, microspheres and nanospheres. See Reddy, Ann. Pharmacother., 34(7-8):915-923 (2000)).

### Exemplary Uses of the Mutable Collagenous Tissue of the Present Technology

The Mutable Collagenous Tissue of the present technology, is a unique natural extracellular matrix composed of collagen type I, and glycosaminoglycan, which is bound to the collagen type I. This structure creates an optimal environment for new collagen biosynthesis, promoting integrin binding, which impart the necessary pharmacological properties helpful for the pharmacological and cosmetic applications disclosed herein. The Mutable Collagenous Tissue of the present technology is biodegradable and exhibits weak antigenicity, making it a suitable for biomedical applications.

The following properties of the Mutable Collagenous Tissue of the present technology, significantly increase the effectiveness of the Mutable Collagenous Tissue of the present technology in wound healing: biocompatibility, ability to promote of cell attachment, physical and chemical stability, improved mechanical properties, and the antimicrobial, and hemostatic properties of the glycosaminoglycan component present in the Mutable Collagenous Tissue of the present technology.

The Mutable Collagenous Tissue of the present technology is useful in methods relating to the wound healing, tissue repair and the treatment of conditions associated to chronic wounds, diabetic wounds, etc. In one aspect, the present technology relates to the treatment of a wound in a subject in need thereof through the administration of therapeutically effective amounts of a wound healing formulation comprising the Mutable Collagenous Tissue of the present technology. In some embodiments, the methods enhance cell attachment and proliferation, or promotion of cell-specific morphology and function. In some embodiments, the wound deals damage to one or more cell type, including but not limited to endothelial cells, hepatocytes, and muscle cells.

In some embodiments, the wounds to be treated include, but are not limited to, an incision wound, a chop wound, a puncture wound, a contused wound, a laceration wound, an impalement injury, dermabrasion, decollement, an abrasion, a crushed wound, contusion, a firearm wound, a blast injury, a biting wound, excoriation, an explosion injury, a bruise, an external wound, a bedsore, a surgical wound, a burn wound, a gunshot wound, subcutaneous abscess, sutured tear, contaminated tear, stasis ulcer, leg ulcer, foot ulcer, venous ulcer, diabetic ulcer, ischemic ulcer, pressure ulcer, an acute/chronic wound, an open injury and a closed injury. In some embodiments, the wound is a chronic wound or a diabetic wound.

In some embodiments, the Mutable Collagenous Tissue of the present technology is formulated as a wound healing formulation, including but not limited to, traditional dressing, antimicrobial dressing, anti-inflammatory and analgesic dressing, wound-drug delivery, medicated suture, or tissue-engineered skin substitute. In some embodiments, the Mutable Collagenous Tissue of the present technology is formulated as a wound healing formulation, including but not limited to, a lotion, ointment, gel, cream, liquid, emulsion, suppository, drop, spray, hydrogel, dressing, bioadhesive gel, aerosol, paste, foam, sunscreen, or powder.

The amount of Mutable Collagenous Tissue of the present technology in the wound healing formulation may be about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 22 wt. %, about 24 wt. %, about 26 wt. %, about 28 wt. %, about 30 wt. %, about 32 wt. %, about 34 wt. %, about 36 wt. %, about 38 wt. %, about 40 wt. %, about 42 wt. %, about 44 wt. %, about 45 wt. %, or any range including and/or in between any two of these values. In some embodiments, the formulations of the Mutable Collagenous Tissue of the present technology may include additional ingredients, including, but not limited to, natural inert polymers, natural bioactive polymers, and synthetic polymers.

The wound healing formulation may include about 0.001 wt. % to about 5 wt. % of a wound healing promoter. Exemplary wound-healing promoters include, but are not limited to, an anti-inflammatory drug (e.g., acetaminophen), zinc, zinc oxide, an inorganic zinc salt (e.g., ZnCl2), a zinc complex, vitamin E, vitamin C, a fibroblast growth factor, a platelet-derived growth factor (see, e.g., Gowda, Santosh, et al. “Topical application of recombinant platelet-derived growth factor increases the rate of healing and the level of proteins that regulate this response.” International Wound Journal 12.5 (2015)^{.} 564-57), or a combination of any two or more thereof. The total amount of wound healing promoter(s) in wound healing formulation may be about 0.001 wt. %, about 0.002 wt. %, about 0.003 wt. %, about 0.004 wt. %, about 0.005 wt. %, about 0.006 wt. %, about 0.007 wt. %, about 0.008 wt. %, about 0.009 wt. %, about 0.01 wt. %, about 0.02 wt. %, about 0.03 wt. %, about 0.04 wt. %, about 0.05 wt. %, about 0.06 wt. %, about 0.07 wt. %, about 0.08 wt. %, about 0.09 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, or any range including and/or in between any two of these values.

In any embodiment disclosed herein, the wound healing formulation may include a solvent and an effective amount of at least one agent selected from the group consisting of a growth factor, an antimicrobial agent, a peptide, a biopolymer, a binder, a colorant, a deodorant agent, a filler, a plasticizer, a viscosity modifier, or any combination thereof. Additionally, or alternatively, in some embodiments, the solvent may be an aqueous solvent or an organic solvent. Additionally, or alternatively, in some embodiments, the aqueous solvent may include a dilute organic acid, for example, acetic acid, citric acid, or a combination thereof.

In any embodiment disclosed herein, the antimicrobial agent may include one or more of polyhexamethylene biguanide, acetic acid, benzoic acid, povidone iodine, natamycin, nisin, citric acid, sorbic acid, propionic acid, honey, sulfites, or any combination thereof. In any embodiment disclosed herein, the peptide may be one or more of defensins, histatins, cathelicidin LL-37, or any combination thereof.

The Mutable Collagenous Tissue of the present technology is useful in methods relating to cosmetics and skin health. Accordingly, in one aspect, the present technology relates to the treatment of skin in a subject in need thereof, through the administration of cosmetically effective amounts of a cosmetic composition comprising the Mutable Collagenous Tissue of the present technology. In some embodiments, the methods include, but are not limited to, reducing skin wrinkles, enhancing skin moisture content, reducing skin moisture evaporation, inhibiting erythema, and suppressing the thickening of skin epidermis layers, exfoliating skin, enhancing skin elasticity, inhibiting erythema, and/or alleviating skin photoaging. In some aspects, the cosmetic composition is topically applied. In some embodiments, the cosmetic composition comprising the Mutable Collagenous Tissue of the present technology is formulated as a lotion, ointment, gel, cream, liquid, paste, drop, spray, hair restorer, lip cream, patch, thickening gel, water-in-oil emulsion, oil-in-water emulsion, solid, sheet, powder, gel, and mousse.

In some embodiments, the amount of Mutable Collagenous Tissue of the present technology present in the cosmetic composition is about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 22 wt. %, about 24 wt. %, about 26 wt. %, about 28 wt. %, about 30 wt. %, about 32 wt. %, about 34 wt. %, about 36 wt. %, about 38 wt. %, about 40 wt. %, about 42 wt. %, about 44 wt. %, about 45 wt. %, or any range including and/or in between any two of these values. In some embodiments, the amount of Mutable Collagenous Tissue of the present technology present in the cosmetic composition is about 0.1 wt. % to about 0.5 wt. %, about 0.25 wt. % to about 2 wt. %, about 1 wt. % to about 5 wt. %, about 2.5 wt. % to about 10 wt. %, about 5 wt. % to about 25 wt. %, or about 10 wt. % to about 50 wt. %.

In some embodiments, the cosmetic composition further comprises cosmetically acceptable carriers or additives selected from the group consisting of moisturizers, antioxidants, cell activators, whitening agents, UV inhibitors, active oxygen removing agents, metal sequestrants, anti-inflammatory agents, antihistamines, vitamins, plant extracts, skin astringents, skin function-enhancing agents, disinfectants, oils, solvents, buffers, bulking agents, anti-microbial agents, thickeners, antioxidants, surfactants, emulsifiers, solubilizers, solubilizing aids, colorants (dyes and pigments), perfumes, and preservatives.

Suitable cosmetically acceptable carriers or additives are glycerol, 1,3-butylene glycol, proteins or their derivatives or hydrolyzates (e.g., elastin or keratin, etc.), mucopolysaccharides and their derivatives (e.g., hyaluronic acid, hydrolyzed hyaluronic acid, chondroitin sulfate, etc.), amino acids and their derivatives (e.g., histidine, serine, glycine, theanine, aspartic acid, arginine, lysine, pyrrolidone carboxylic acid, N-methyl-L-serine, etc.), carbohydrates (e.g., sorbitol, erythritol, trehalose, inositol, glucose, xylitol, N-acetyl glucosamine, raffinose, sucrose and its derivatives, dextrin and its derivatives, honey, etc.), phosphorylated oligosaccharides and their mineral salts, D-panthenol and its derivatives, glycolipids, ceramides, glycosyl ceramides, sweet Hydrangea leaf extract, almond extract, angelica extract, avocado extract, Althaea officinalis extract, Arnica extract, hot spring water, Citrus peel extract, aloe extract, Malva sylvestris extract, Scutellaria baicalensis extract, Coptidis rhizoma extract, St. John's wort extract, Lamium album extract, Ononis extract, chamomile extract, oats extract, glabridin, glabrene, liquiritin, isoliquiritin and licorice extracts containing the same, water-soluble and oil-soluble licorice extracts, bramble extract, yellow Himalayan raspberry extract, honeysuckle (Lonicera japonica Thunb) extract, quince seed (Cydonia oblonga) extract, Sophora flavescens extract, gardenia extract, Sasa veitchii extract, water-soluble chlorophyll, maple leaf extract, Citrus extract, grapefruit extract, watercress extract, Gentiana scabra extract, geranium herb extract, burdock extract, sesame extract, wheat extract, wheat germ extract, comfrey (Symphytum officinale) extract, Asiasari radix extract, cactus extract, Saponaria officinalis extract, salvia (sage) extract, hawthorn extract, Rehmannia glutinosa extract, perilla extract, herb Robert extract, meadow sweet extract, Paeoniae radix extract, ginger extract, Japanese iris extract, white birch extract, mentha (peppermint, Mentha spicata, spearmint, etc.) extracts, Malva (marshmallow) extract, Equisetum arvense extract, Cnidium rhizome extract, Morus alba extract, Thymus vulgaris extract, camellia extract, Angelica acutiloba extract, plant worm extract, corn extract, Houttuynia cordata extract, hibiscus extract, white willow extract,Potentilla tormentilla extract, parsley extract, Job's tears (coix seed) extract, Hamamelis virginiana (witch-hazel) extract, rose extract, cypress extract, sunflower extract, Tussilago farfata extract, butcher's broom extract, grape extract, prune (Japanese plum) extract, sponge gourd extract, avocado extract, okra extract, linden extract, Alpinia speciosa leaf extract, Paeonia suffruticosa extract, hop extract, jojoba leaf extract, jojoba oil, macadamia nut oil, olive oil, apricot-kernel oil, persic oil, safflower oil, sunflower seed oil, avocado oil, camellia oil, almond oil, perilla oil, sesame oil, borage (Borago officinalis) oil, cacao butter, shea butter, pine extract, horse chestnut extract, Sapindus mukorossi extract, mucin, Lithospermi extract, meadowfoam oil, Melissa extract, cornflower extract, Saxifraga stolonifera extract, lily extract, lime extract, lavender extract, apple extract, Gentiana scabra extract, phospholipids (from soybean, egg yolk, etc.), milk vetch extract, Sanguisorba officinalis extract, tea (oolong tea, green tea, black tea, etc.) extracts, Akebia stem extract, urea, Siraitia grosvenorii extract, white fungus polysaccharide, seaweed extracts (brown algae such as Laminariaceae Bory, Saccharina japonica, Undaria pinnatifida, Sargassum fusiforme, Fucus vesiculosus, Costaria costata, Saccharina gyrata, Ecklonia cava, Ecklonia stolonifera, Alaria crassifolia Kjellman, Sargassum fukvellum, and giant kelp); red algae such as Ceylon moss, Kappaphycus striatum, Eucheuma denticulatum, Chondrus ocellatus Holmes, Chondracanthus tenellus, Nithophyllum, Pyropia tenera, Polyopes affinis, Grateloupia crispata, Gloiopeltis tenax, Gracilaria vermiculophylla, Ceratodictyon spongiosum, Ceramium kondoi, and Campylaephora hypnaeoides; green algae such as chlorella, green laver, Dunaliella Chlorococcales, Ulva pertusa, Prasiola japonica, Aegagropila linnaei, Cladophoraceae, Acetabularia ryukyuensis, Chaetomorpha crassa, Chaetomorpha moniligera, Monostroma nitidum, and Spirogyra; blue green algae such as spirulina), plant fermented liquids such as pear juice fermented liquid, hibiscus fermented liquid, rice fermented liquid, vitamin E and its derivatives, vitamin A and its derivatives, carotenoids (e.g., carotene, lycopene, astaxanthin, capsanthin, etc.), vitamin B (e.g., thiamine hydrochloride, thiamine sulfate, riboflavin, riboflavin acetate, pyridoxine hydrochloride, pyridoxine dioctanoate, flavin adenine nucleotide, cyanocobalamine, folic acids, nicotinamide, benzyl nicotinate and other nicotinic acids, cholines, etc.), ascorbic acid and its derivatives, vitamin D and its derivatives, mevalonolactone, rutin and its derivatives, thiotaurine, taurine, hydroquinone and its derivatives, catechin and its derivatives, glabridin, glabrene, liquiritin, isoliquiritin and licorice extracts containing the same, glutathione and its derivatives, gallic acid and its derivatives, Pycnogenol, bitter orange peel extract, Oenothera biennis extract, cucumber extract, Millettia reticulata extract, cholesterol and its derivatives, superoxide dismutase, Ginkgo biloba extract, ginseng extract, Rosa maikwai extract, Chaenomeles sinensis extract, Alnus firma extract, stevia extract, lettuce extract, tea extracts (oolong tea, black tea, green tea, etc.), microorganism fermentation metabolites, Ganoderma lucidum extract, eggshell membrane extract, placenta extract, Siraitia grosvenorii extract.

In some embodiments, the cosmetic composition comprises ingredients one or more selected from the group consisting of the Mutable Collagenous Tissue of the present technology, organic aloe leaf juice (Aloe barbadensis), coconut oil (Cocos nucifera), apricot kernel oil (Prunus armeniaca), glycerin, emulsifying wax (e.g., cetyl alcohol, stearyl alcohol, polysorbate 60), stearic acid, vitamin E (Tocopherol), sunflower seed oil (Helianthus annuus), phenoxyethanol, glycolic acid, tartaric acid, malic acid, organic shea butter (Butyrospermum parkii), mango seed butter (Mangifera indica), organic sugar cane extract (Saccharum officinarum), organic bilberry fruit extract (Vaccinium myrtillus), organic sugar maple extract (Acer saccharinum), organic orange peel extract (e.g., Citrus sinensis), organic lemon peel extract (Citrus limon), organic cranberry fruit extract (Vaccinium macrocarpon), organic seaweed extract (Fucus vesiculosus), organic green tea leaf extract (Camellia sinensis), organic white willow bark extract (Salix alba), organic alcohol, organic neem seed oil (Melia azadirachta), organic rosemary leaf extract (Rosmarinus officinalis), xanthan gum, tetrasodium glutamate diacetate, tranexamic acid, and astaxanthin. In some embodiments, the cosmetic composition comprises ingredients one or more selected from the group consisting of Mutable Collagenous Tissue of the present technology, organic aloe leaf juice, organic coconut oil, organic alcohol, cetearyl olivate, sorbitan olivate, cetyl palmitate, sorbitan palmitate, organic olive fruit oil (Olea europaea), stearic acid, organic green tea leaf extract (Camellia sinensis), pomegranate seed extract (Punica granatum), xanthan gum, soluble collagen, ascorbic acid, tocopheryl acetate, royal jelly, and astaxanthin. In some embodiments, the cosmetic composition comprises ingredients one or more selected from the group consisting of the Mutable Collagenous Tissue of the present technology, water, organic coconut oil, grapeseed oil (Vitis vinifera), emulsifying wax (e.g., cetyl alcohol, stearyl alcohol, polysorbate 60), stearic acid, glycerin, xanthan gum, phenoxyethanol, tetrasodium glutamate diacetate, soluble collagen, ascorbic acid, tocopheryl acetate, royal jelly, and astaxanthin.

In some embodiments, the cosmetic composition intensely hydrates skin to create a softer and smoother look, reducing the appearance of scars. In some embodiments, the composition hydrates and soothes skin to create a visibly supple texture around the eye, diminishing the appearance of puffiness, temporary redness and uneven skin tone. In some embodiments, the cream revitalizes skin on contact and helps improve skin appearance. In some embodiments, the composition promotes smooth and hydrated skin, reducing the appearance of dark spots and hyperpigmentation for a more radiant look.

In one aspect, the present technology relates to hydrogel compositions comprising the Mutable Collagenous Tissue of the present technology for injection. The hydrogel compositions and methods may be applied to subjects seeking a small/moderate enlargement, shape change or contour alteration of a body part or region. The conditions that are treated with the hydrogel compositions and methods include, but are not limited to, breast imperfections, e.g., a breast augmentation, a breast reconstruction, defects due to implant complications; a facial imperfections, e.g., a facial augmentation, wrinkles, lines, a facial reconstruction, scars, sunken cheeks, hollow cheeks, thin lips, nasal imperfections or defects, retro-orbital imperfections or defects, a facial folds, nasolabial lines, perioral lines, and/or a marionette line; skin imperfections, e.g., an augmentation or a reconstruction of the upper arm, lower arm, hand, shoulder, back, neck, torso including abdomen, buttocks, upper leg, lower leg including calves, foot including plantar fat pad, eye, genitals, or other body part, region or area. In some embodiments, the method comprises intra-epidermal, intra-dermal, and/or subcutaneous injection of cosmetically sufficient amount of the hydrogel compositions. In some embodiments, the cosmetically sufficient amounts of the hydrogel composition are determined based on the alteration and/or improvement desired, the reduction and/or elimination of a soft tissue condition symptom desired, the clinical and/or cosmetic effect desired by the individual and/or physician, and the body part or region being treated. In some embodiments, the injection of cosmetically sufficient amount of the hydrogel compositions causes one or more of gain of skin elasticity and recoil, improved barrier function, decreased transepidermal water loss, desired small/moderate enlargement, desired shape change or contour alteration, desired, reduction of wrinkles, firming of skin, etc. In some embodiments, the hydrogel compositions promote cartilage regeneration. In some embodiments, the hydrogel compositions are used for the treatment of osteoarthritis.

In some embodiments, the amount of Mutable Collagenous Tissue of the present technology present in hydrogel composition is about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, about 16 wt. %, about 17 wt. %, about 18 wt. %, about 19 wt. %, about 20 wt. %, about 22 wt. %, about 24 wt. %, about 26 wt. %, about 28 wt. %, about 30 wt. %, about 32 wt. %, about 34 wt. %, about 36 wt. %, about 38 wt. %, about 40 wt. %, about 42 wt. %, about 44 wt. %, about 45 wt. %, or any range including and/or in between any two of these values. In some embodiments, the amount of Mutable Collagenous Tissue of the present technology present in the hydrogel composition is about 0.1 wt. % to about 0.5 wt. %, about 0.25 wt. % to about 2 wt. %, about 1 wt. % to about 5 wt. %, about 2.5 wt. % to about 10 wt. %, about 5 wt. % to about 25 wt. %, or about 10 wt. % to about 50 wt. %.

In some embodiments, the amount of the hydrogel composition administered is, e.g., about 0.01 g, about 0.05 g, about 0.1 g, about 0.5 g, about 1 g, about 5 g, about 10 g, about 20 g, about 30 g, about 40 g, about 50 g, about 60 g, about 70 g, about 80 g, about 90 g, about 100 g, about 150 g, or about 200 g. In some embodiments, the amount of a hydrogel composition administered is, e.g., about 0.01 g to about 0.1 g, about 0.05 g to about 0.5 g, about 0.1 g to about 1 g, about 0.5 g to about 5 g, about 1 g to about 10 g, about 5 g to about 50 g, about 10 g to about 100 g, or about 50 g to about 200 g. In some embodiments, the amount of a hydrogel composition administered is, e.g., about 0.01 mL, about 0.05 mL, about 0.1 mL, about 0.5 mL, about 1 mL, about 5 mL, about 10 mL, about 20 mL, about 30 mL, about 40 mL, about 50 mL, about 60 mL, about 70 g, about 80 mL, about 90 mL, about 100 mL, about 150 mL, or about 200 mL. In some embodiments, the amount of a hydrogel composition administered is, e.g., about 0.01 mL to about 0.1 mL, about 0.05 mL to about 0.5 mL, about 0.1 mL to about 1 mL, about 0.5 mL to about 5 mL, about 1 mL to about 10 mL, about 5 mL to about 50 mL, about 10 mL to about 100 mL, or about 50 mL to about 200 mL.

In one aspect, the present technology relates to nutritional supplements comprising the Mutable Collagenous Tissue of the present technology. In some embodiments, the nutritional supplement is formulated as powder, pill, tablet, capsule, beverage, frozen dessert, gelatin dessert, pudding, confectionery, soft gel, chewing gum, candy, gummi candy, gum, caramel, chocolate, tablet sweet, snack, baked good, jelly, jam, yogurt, soup, or stew. The nutritional supplement may further comprise moisturizers, antioxidants, minerals, metal sequestrants, proteins, enzymes, vitamins, fats, oils, dietary fiber, fruit extracts, vegetable extracts, meat extracts, yeast extract, solvents, buffers, bulking agents, anti-microbial agents, thickeners, antioxidants, surfactants, emulsifiers, solubilizers, solubilizing aids, colorants (dyes and pigments), perfumes, flavoring agents, sugars, and preservatives.

In some embodiments, the nutritional supplement promotes joint health, supports connective tissues, helps maintain a strong immune system, supports restful sleep and/or healthy digestion. In some embodiments, the nutritional supplement corrects nutritional insufficiency. In some embodiments, the nutritional supplement stimulates testosterone production to maintain healthy libido, stamina and sexual performance.

In one aspect, the present technology relates to use of the Mutable Collagenous Tissue of the present technology, or any of the compositions disclosed herein, as an extracellular matrix for cell culture and tissue engineering. In one aspect, the present technology relates to use of the Mutable Collagenous Tissue of the present technology, or any of the compositions disclosed herein, as bio-ink for electrospinning and 3D-printing.

### Kits and Articles of Manufacture

In one aspect, the present disclosure provides a kit comprising a mutable collagenous tissue purified according to any of the methods disclosed herein, and/or any of the compositions disclosed herein. In some embodiments, the kits comprise the mutable collagenous tissue and/or the compositions that are packed in suitable containers, or a dispenser device. In some embodiments, the kits contain the composition along with instructions for use. In some embodiments, the kits these components may be stored in unit or multi-dose containers, for example, sealed ampoules, vials, bottles, syringes, and test tubes, as an aqueous, preferably sterile, solution or as a lyophilized, preferably sterile, composition for reconstitution. The kit may further comprise a second container which holds a diluent suitable for diluting the composition towards a higher volume. Suitable diluents include, but are not limited to, a pharmaceutically acceptable excipient of the composition and a saline solution. Furthermore, the kit may comprise instructions for diluting the composition and/or instructions for administering the composition, whether diluted or not. The containers may be formed from a variety of materials such as glass or plastic and may have a sterile access port (for example, the container may be an intravenous solution bag or a vial having a stopper which may be pierced by a hypodermic injection needle). The kit may further comprise more containers comprising a pharmaceutically acceptable buffer, such as phosphate-buffered saline, Ringer's solution and dextrose solution. It may further include other materials desirable from a commercial and user standpoint, including other buffers, diluents, filters, needles, syringes, culture medium for one or more of the suitable hosts. The kits may optionally include instructions customarily included in commercial packages of therapeutic or cosmetic products, that contain information about, for example, the indications, usage, dosage, manufacture, administration, contraindications and/or warnings concerning the use of such therapeutic or cosmetic products.

The kit can also comprise, e.g., a buffering agent, a preservative or a stabilizing agent. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present technology may contain a written product on or in the kit container. The written product describes how to use the reagents contained in the kit. In certain embodiments, the use of the reagents can be according to the methods of the present technology.

In some embodiments, the kit includes a container comprising the compositions of the present disclosure. The container may be light impermeable, air-tight and/or leak resistant. Exemplary containers include, but are not limited to, syringes, vials, or pouches. In some embodiments, the kit comprises a handheld injection device.

In one aspect, the present disclosure provides a prefilled syringe comprising hydrogel comprising the Mutable Collagenous Tissue of the present technology.

In one aspect, the present disclosure provides applicators for applying the Mutable Collagenous Tissue of the present technology. In some embodiments, the applicator is selected from sponge, puff, tube, application nozzle, wipes, spray, a spreadable stick, pencil, and discharge pad. The applicator may be part of a kit for preparing and/or administering any of the compositions of the present disclosure.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Extraction of Mutable Collagenous Tissue from Sea Urchin

The following method was used to extract mutable collagenous tissue from sea urchin:

Step 1: Sea urchin specimens (Strongylocentrotus purpuratus and/or Paracentrotus lividus) were collected and stored at −20° C. for transportation. The sea urchin specimens were thawed and dissected to eliminate internal tissue. Connective tissue from the body wall was isolated.

Step 2: Connective tissue from Step 1 was soaked in aqueous isopropyl alcohol (50% v/v) solution for 30 minutes at 4° C., cut into small pieces and stored at −20° C. for 12 hours.

Step 3: The frozen connective tissue was then soaked in ice-cold deionized water at 4° C. for 30 minutes, and centrifuged at 4,500×g at 4° C. for 15 minutes. The supernatant was discarded.

Step 4: The connective tissue from the pellet of Step 3 (50 g) was then suspended in a 2-liter ice-cold, aqueous solution of 1 μM EGTA in PBS buffer and incubated at 4° C. for 12 hours with gentle stirring. The mixture was then centrifuged at 4,500×g at 4° C. for 30 minutes, and the supernatant was discarded.

Step 5: Solid connective tissue from the pellet of Step 4 was dispersed in 2 liters of an ice-cold, aqueous 1.0 M sodium bicarbonate solution and incubated at 4° C. with gentle stirring for 24 hours. The mixture was centrifuged at 4,500×g, 4° C. for 30 minutes, and the supernatant was discarded.

Step 6: The pellet from Step 5 of Example 1 was suspended in a 0.5 M citric acid solution containing trypsin (0.5% w/v) and reacted at 25° C. with stirring for 48 hours. The reaction mixture was subjected to centrifugation at 9,000×g, 4° C. for 60 minutes. The supernatant is the extracted mutable collagenous tissue.

### Example 2: Extraction of Mutable Collagenous Tissue from Sea Cucumber

The following method was used to extract mutable collagenous tissue from sea cucumber:

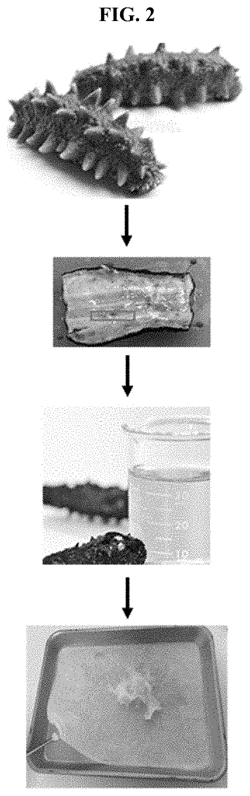

Step 1: Sea cucumber specimens (Thelenota ananas and/or Cucumaria frondosa) were collected and stored at −20° C. for transportation. As shown in FIG. 2, the sea cucumber specimens were thawed and dissected to eliminate internal tissue. Connective tissue from the body wall isolated.

Step 2: Connective tissue from Step 1 were then soaked in ice-cold, aqueous ethanol (45% v/v) solution for 30 minutes, cut into small pieces and stored at −20° C. for 12 hours.

Step 3: Frozen connective tissue from Step 2 were soaked in deionized water for 30 minutes, centrifuged at 9,000×g, 4° C. for 30 minutes and the supernatant was discarded.

Step 4: Connective tissue from the pellet of Step 3 (50 g) was suspended in a 2-liter aqueous solution of 0.8 μM EDTA in 10 mM Tris buffer and mixed for 24 hours under gentle stirring. After the reaction time is completed the mixture is centrifuged at 9,000×g, 4° C. for 30 minutes and the supernatant is discarded.

Step 5: Solid connective tissue from Step 4 was dispersed in an aqueous 0.1 M sodium hydroxide solution and incubated with gentle stirring at 4° C. for 48 hours. The resultant mixture was subjected to centrifugation at 9,000×g, 4° C. for 30 minutes, and the supernatant was discarded.

Step 6: Remaining collective tissue from Step 5 was suspended in an acetic acid solution (0.1 M) containing pepsin (0.5% w/v) and reacted with stirring for 72 hours. The reaction mixture was subjected to centrifugation at 12,000×g at 4° C. for 60 minutes. The supernatant is the purified mutable collagenous tissue (See the solution in the beaker in FIG. 2).

### Example 3: Extraction of Solid Collagen Type I from Sea Urchin

Extraction of native collagen from sea urchin was performed using the following steps:

Step 1: Extracted mutable collagenous tissue solution in 0.5 M citric acid from (See Example 1, Step 6, supra) was mixed with 0.5 M potassium chloride at a 1:5 molar ratio, to induce protein precipitation. The mixture was then separated by centrifugation at 4,500×g, 4° C. for 60 minutes. The supernatant is discarded.

Step 2: Protein precipitate from Step 1 was dissolved in of 0.5 M citric acid solution and dialyzed against deionized water at 4° C. for 72 hours using cellulose membranes with 10 kDa molecular weight cut-off to obtain mutable collagenous tissue solution.

Step 3: Native mutable collagenous tissue solution from Step 2 was be frozen at −20° C. and subjected to freeze drying to obtain a sheet of mutable collagenous tissue from sea urchin, which had a fibrous character similar to collagen type I (data not shown). The mutable collagenous tissue collagen sheet was ground to prepare mutable collagenous tissue powder.

Step 4: to determine content and purity of the mutable collagenous tissue, it dissolved in 0.1 M acetic acid, resolved using SDS-PAGE. The gels were stained using Coomassie blue stain. As shown in FIG. 3 (MC-2), the mutable collagenous tissue contained protein bands at and above 150 kDa. Compared to commercially available pure samples of collagen type I from bovine, rat and human tissue (collectively, the controls), the purified sea urchin mutable collagenous tissue of the current disclosure did not show bands of impurities/degradation products below 150 kDa. Moreover, as shown in FIG. 3 (MC-2), compared to the controls, the purified sea urchin mutable collagenous tissue featured higher levels of bands corresponding to α1, α2 chains, as well as β bands. This pattern is consistent with the purified sea urchin mutable collagenous tissue being collagen type I. Densitometric analysis showed that the purified sea urchin collagen type I was more than 98% pure (data not shown).

### Example 4: Extraction of Solid Collagen Type I from Sea Cucumber

Extraction of native collagen from sea urchin was performed using the following steps:

Step 1: Extracted mutable collagenous tissue solution in 0.1 M acetic acid (See FIG. 2 (third panel), and Example 2, Step 6, supra) was mixed with sodium chloride (0.1 M) to induce protein precipitation. The mixture was subjected to centrifugation at 9,000×g, 4° C. for 60 minutes. The supernatant was discarded.

Step 2: Protein precipitate from Step 1 was dissolved in 5 ml ice-cold 0.1 M acetic acid and dialyzed against deionized water at 4° C. for 48 hours using cellulose membranes with 10 kDa molecular weight cut-off to obtain native collagen solution.

Step 3: Native collagen solution from Step 2 was frozen at −20° C. followed by freeze drying to obtain a sheet of mutable collagenous tissue from sea cucumber. As shown in FIG. 1A (lower panel) and FIG. 2 (lower panel), the sheet of mutable collagenous tissue from sea cucumber had a fibrous character similar to collagen type I. The mutable collagenous tissue collagen sheet was ground to prepare mutable collagenous tissue powder.

Step 4: to determine content and purity of the mutable collagenous tissue from sea cucumber, it dissolved in 0.1 M acetic acid, resolved using SDS-PAGE. The gels were stained using Coomassie blue stain. As shown in FIG. 3 (MC-1), the mutable collagenous tissue from sea cucumber contained protein bands at and above 150 kDa. Compared to commercially available pure samples of collagen type I from bovine, rat and human tissue (the controls), the purified mutable collagenous tissue from sea cucumber of the current disclosure did not show bands of impurities/degradation products below 150 kDa. Moreover, as shown in FIG. 3 (MC-1), compared to the controls, the purified sea urchin mutable collagenous tissue featured higher levels of bands corresponding to α1, α2 chains, as well as β bands. This pattern is consistent with the purified sea cucumber mutable collagenous tissue being collagen type I. Densitometric analysis showed that the purified sea cucumber collagen type I was more than 98% pure (data not shown).

### Example 5: Extraction of Solid Collagen Type I from Starfish

Mutable collagenous tissue and solid collagen type I from starfish was purified substantially as disclosed in Examples 1-4.

Mutable collagenous tissue from starfish was also subjected to SDS-PAGE. As shown in FIG. 3 (MC-3), the mutable collagenous tissue from starfish contained protein bands at and above 150 kDa. Compared to commercially available pure samples of collagen type I from bovine, rat and human tissue (the controls), the purified mutable collagenous tissue from starfish of the current disclosure did not show bands of impurities/degradation products below 150 kDa. Moreover, as shown in FIG. 3 (MC-3), compared to the controls, the purified starfish mutable collagenous tissue featured higher levels of bands corresponding to α1, α2 chains, as well as β bands. This pattern is consistent with the purified sea urchin mutable collagenous tissue being collagen type I. Densitometric analysis showed that the purified sea cucumber collagen type I was more than 98% pure (data not shown).

### Example 6: Evaluation of Collagen Properties and Purity

Collagen content and purity was evaluated by SDS-PAGE. Collagen samples were dissolved in acetic acid (0.1 M) to a final concentration of 1 mg/mL and mixed 1:1 with Load Running Buffer (Bio Rad, USA). Running buffer (1×) was prepared from concentrated 10× buffer stock solution Tris-Glycine-SDS (Bio Rad, USA). Liquid samples were filtered through a 0.45 μm filter and 7 μL were pipetted into each designated well on a pre-casted polyacrylamide 7.5% Mini-Protean TGX gels (Bio Rad, USA). Gel electrophoresis was conducted in a Bio Rad Mini-Protean Tetra SDS-PAGE chamber (Bio Rad, USA) under at 200 V, for about 40 min, until the blue marker run out of the gel. Proteins present in the gel were stained with Coomassie blue using standard protocol. The gel was imaged and subjected to densitometry.

As shown in FIG. 3, commercially available human type-I collagen (Advanced Biomatrix, USA), rat tail type-I collagen (Sigma Aldrich, USA) and bovine type-I collagen (Sigma Aldrich, USA) exhibited protein bands in 15 kDa to 100 kDa region, which are likely to be the degradation products of collagen, or small protein- or peptide-contaminants that were not removed by the extraction process. In comparison, the samples MC-1 through MC-3 showed high molecular weight collagen protein bands, which appeared between 150 kDa and 300 kDa. These protein bands correspond to pure native collagen protein in its intact form. Based on molecular weights, distinctive signals for collagen type I were assigned to α1, α2 and β-collagen, corresponding to the typical triple helix chains found in native collagen macrostructure.

These data show that the method disclosed herein produces pure collagen type I from echinoderms. The method disclosed herein does not lead to either chemical (acid/base) or enzymatic (proteases) degradation, producing pure high molecular weight collagen type I (See FIG. 3).

### Example 7: Analysis of Purity and Stability

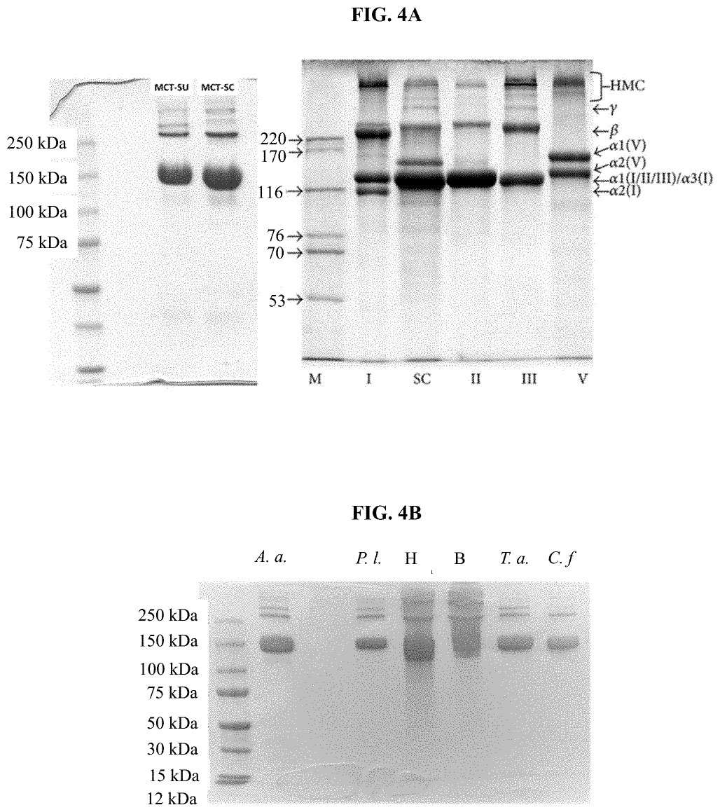

Collagen samples were subjected to electrophoretic characterization of collagen samples. Native collagen types I, II, III and V were used as controls for comparison. Collagen type I collagen contains, (α1)(I)₂(α2)(I)₁. As shown in FIG. 4A, echinoderm mutable collagenous tissue corresponded with the typical profile of pure collagen type I, as evidenced from the molecular profile study. For example, SDS-PAGE pattern exhibited an intense band near 100 kDa followed by a faint band, which correspond to α1 and α2 chains, respectively. In addition, a band observed near 200 kDa corresponds to β-chain (dimers of α1-chains; data not shown).

To confirm whether MCT from other sources also is collagen type I, SDS-PAGE was performed. As shown in FIG. 4B, for native collagen samples showed that mutable collagenous tissue samples from Asterias amurensis, Paracentrotus lividus, Thelenota ananas, and Cucumaria frondosa, isolated by methods described above, showed a mobility pattern showing both α1 and α2 chains. The band of al-chain was about twice as intense as that of α2-chain, consistent with the molecular formula (α1)(I)₂(α2)(I)₁ (See FIG. 4B). Other cross-linked subunits with high molecular weight, such as β-chain (dimers of α1-chains) were also visible. On the basis of subunit composition and SDS-PAGE pattern, it was interpreted that echinoderm MCT is mainly composed of collagen type I, which was consistent with the human and bovine collagen type I collagen.

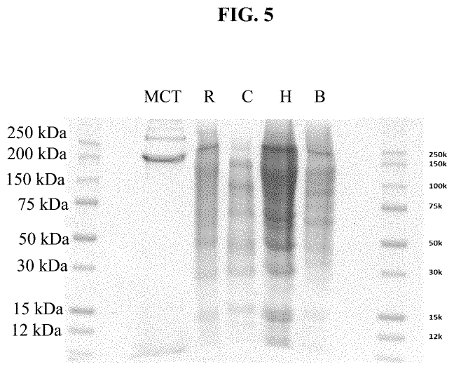

To understand stability of collagen during extraction process, SDS-PAGE was performed under reducing conditions by adding β-mercaptoethanol to the sample buffer to promote hydrolysis of disulfide linkages. A sample of sea cucumber MCT was compared with rat, chicken, human and bovine collagen type I samples obtained from commercial sources. The rat, chicken, human and bovine collagen type I samples served as reference samples. As shown in FIG. 5, MCT collagen showed no bands under 150 kDa indicating that the sea cucumber collagen was intact. In contrast, the collagen reference samples showed multiple peptide bands along gel, indicating existence of chemical and/or enzymatic degradation during extraction. Sea cucumber MCT collagen showed its native collagen bands as shown in the previous SDS-PAGE analysis under native conditions. Accordingly, the MCT obtained by the process described herein protects the collagen from enzymatic and chemical degradation.

These data show that the method disclosed herein produces pure collagen type I from echinoderms that does not show chemical (acid/base) or enzymatic (proteases) degradation.

### Example 8: Analysis of Chemical Profile

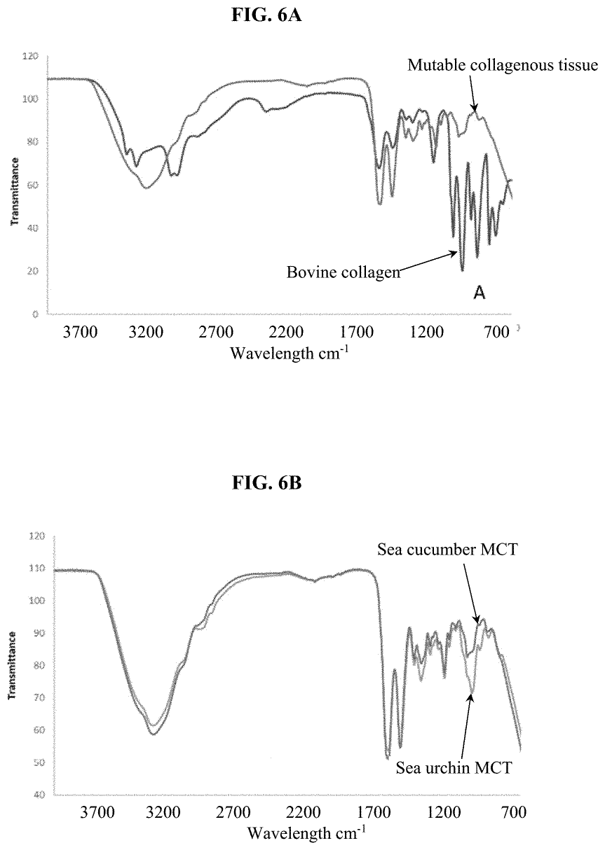

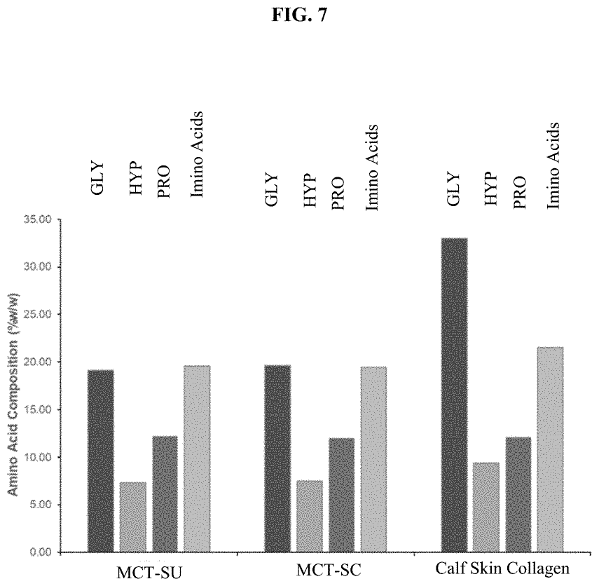

To further characterize sea cucumber mutable collagenous tissue, chemical profile of sea cucumber MCT was studied using Fourier-Transform Infrared Spectroscopy in the attenuated reflectron mode (FTIR-ATR). The FTIR analysis method, which uses infrared light to scan test samples and observe chemical properties, is an analytical technique used to identify organic, polymeric, and, in some cases, inorganic materials. Each molecule or chemical structure produces a unique spectral fingerprint, making FTIR analysis a great tool for chemical identification. FTIR spectra per collagen type could be obtained by using a combination of four spectral intervals [ν(C═O) absorption of amide I (1,700-1,600 cm⁻¹), δ(CH₂), and δ(CH₃) absorptions (1,480-1,350 cm⁻¹), ν(C—N), and δ(N—H) absorptions of amide III (1,300-1,180 cm⁻¹), and ν(C—O) and ν(C—O—C) absorptions of carbohydrate moieties (1,100-1,005 cm⁻¹)] As shown in FIG. 6A, the FTIR spectrum of the bovine collagen exhibits a characteristic chemical profile for collagens, and sea cucumber mutable collagenous tissue also confirms with the typical profile of collagen type I. The slight differences in the FTIR profile of sea cucumber mutable Pcollagenous tissue, compared to bovine collagen I are likely to be associated to different amino acid distributions and triple helix arrangements. Since FTIR analysis only shows the general chemical composition of collagen, no significant differences were expected. To find in the comparative analysis of different MCT samples isolated from sea urchin and sea cucumber, FTIR-ATR analyses were performed. As shown in FIG. 6B, sea urchin and sea cucumber MCT showed very comparable profiles to each other, which included most of the characteristic FTIR peaks typically found in collagen type I. These data also show that the extraction process for mutable collagenous tissue disclosed herein produces for homogeneous echinoderm collagen type I.

To further elucidate the basis for the slight differences in FTIR profiles, amino acid composition of echinoderm mutable collagenous tissue from sea cucumber and sea urchin was analyzed. Bovine collagen isolated from calf skin was used as a standard. As shown in Table 1, the amino acid composition of echinoderm mutable collagenous tissue from sea cucumber and sea urchin was very similar to each other. However, there were slight differences between bovine collagen isolated from calf skin and echinoderm mutable collagenous tissue (See Table 1).

TABLE 1
Amino acid composition of mutable collagenous tissue,
and bovine collagen isolated from calf skin.

| | | MCT-SU | MCT-SC | Calf-skin Collagen |
|---|---|---|---|---|
| Amino Acid | Code | (% w/w) | (% w/w) | (% w/w) |
| CYS | C | 0.00 | 0.00 | 0.00 |
| HYP | Z | 7.33 | 7.46 | 9.40 |
| ASP | D | 9.23 | 8.42 | 4.50 |
| THR | T | 4.37 | 3.38 | 1.80 |
| SER | S | 3.47 | 4.10 | 3.90 |
| GLU | E | 13.46 | 14.01 | 7.50 |
| PRO | P | 12.21 | 11.99 | 12.10 |
| GLY | G | 19.13 | 19.64 | 33.00 |
| ALA | A | 9.22 | 9.99 | 11.90 |
| VAL | V | 2.53 | 2.60 | 2.10 |
| MET | M | 0.65 | 0.61 | 0.60 |
| ILE | I | 1.72 | 1.64 | 1.10 |
| LEU | L | 2.73 | 2.83 | 2.30 |
| TYR | Y | 1.46 | 1.24 | 0.30 |
| PHE | F | 1.42 | 1.19 | 0.30 |
| HIS | H | 0.33 | 0.24 | 0.50 |
| LYS | K | 0.65 | 0.50 | 2.60 |
| ARG | R | 8.54 | 8.65 | 5.10 |

As shown in Table 1, the major amino acids of MCT were glycine (19.0%), glutamic acid (14.0%), proline (12.0%), alanine (9.0%), aspartic acid (9.0%), Arginine (8.0%) and hydroxyproline (6.7%).

The primary structure of type I collagen is characterized as containing domains with continuous repeating of Gly-X-Y sequence (where X is mostly proline and Y is mostly hydroxyproline), and the very short N- and C-terminal regions called telopeptides (15 to 26 amino acid residues). The Gly-X-Y repeating sequence in al chain plays an important role in triple helix formation of secondary structure. Zitnay et al., Acta Biomateralia, 65: 76-87 (2018); Leon-Mancilla et al., Journal of Applied Research and Technology 14: 77-85 (2016). As an amino acid with the lowest molecular weight, glycine residues arranged in the center of triple helix can help helix structure to fold compactly. Cheng et al., Plos One 12(1): e0169731 (2017). Therefore, glycine is the major amino acid in bovine collagen. According to previous references, the glycine content in bovine collagen ranges from 14 to 33%, about one-quarter of total amino acid, that was consistent with MCT glycine content (19%). Imino acids (proline and hydroxyproline) are important amino acids composing Gly-X-Y repeat sequences in a chain, based on that they could maintain the stability of collagen triple helix with their pyrrolidine rings. Nimptsch et al., Cellular Tissue Research 343: 605-617 (2011).

As shown in Table 1, and FIG. 7, The contents of proline and hydroxyproline in MCT were 12.0 and 7.0%, respectively, for a total imino acid content of 20.0%, which was slightly lower than the corresponding values found for bovine collagen. Similarly, as shown in Table 1, and FIG. 7, although bovine collagen had more glycine (33%), less glutamic acid (7.5%) and less aspartic acid (4.5%) compared to the MCT samples, the overall amino acid content of collagen from the different sources were similar to each other.

The contents of proline and hydroxyproline are found to be related to environmental temperature. Zhong et al., Food Chem 80(4): C671-C679 (2015). However, when compared to bovine collagen, the ratio of Gly to imino acid (Hyp/Pro) in MCT content was lower, thus suggesting a more efficient stabilization of the Gly-based triple helix by imino acids in MCT.

### Example 8: Scanning Electron Microscopy

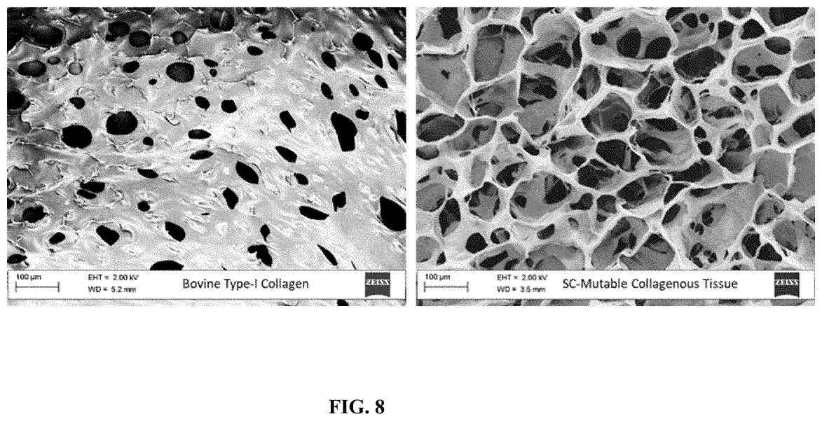

Scanning electron microscopy (SEM) produces images of a sample by scanning the surface with a focused beam of electrons. The electrons interact with atoms in the sample, producing signals that contain information about the sample's surface topography and composition. The surface morphology of mutable collagenous tissue extracted from sea cucumber was characterized by SEM imaging, in comparison with collagen type I from calf skin, used as a reference. As shown in FIG. 8, mutable collagenous tissue exhibited a fibrillar collagen structure similar to that of the reference bovine collagen type I; however, the mutable collagenous tissue showed higher porosity and homogeneity than the reference collagen sample (See FIG. 8). The fibers in the mutable collagenous tissue were bigger and longer compared to the reference. Collagen porosity and pore homogeneity is an important performance parameter for wound dressing applications, due to its direct relation with cell attachment and proliferation.

These data show that the method disclosed herein produces pure collagen type I a pure high molecular weight collagen type I having bigger and longer fibers from echinoderms, which is suitable for the compositions and kits disclosed herein for applications such as wound dressing, cosmetics, nutrition etc.

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for producing a mutable collagenous tissue from an echinoderm, the method comprising treating a connective tissue from an echinoderm with:
(a) an aqueous solution of an organic solvent;
(b) a chelating agent; and
(c) an enzyme solution,
to thereby obtain a liquid mutable collagenous tissue, wherein said mutable collagenous tissue comprises collagen type I and glycosaminoglycan, wherein the glycosaminoglycan is bound to the collagen type I.

2. The method of claim 1, wherein the echinoderms are selected from the group consisting of sea urchins, sea cucumbers, star fish, and a combination thereof.

3. The method of claim 1, wherein the organic solvent is selected from the group consisting of ethanol, isopropanol, acetone, ethyl acetate, and a combination thereof.

4. The method of claim 3, wherein the aqueous solution of the organic solvent comprises about 25% to about 75% organic solvent.

5. The method of claim 1, wherein the chelating agent is selected from the group consisting of ethylenediaminetetraacetic acid (EDTA), ethyleneglycol-bis-(beta-aminoethylether) N,N,N′,N″-tetraacetic acid (EGTA), diethylenetriaminepentaacetic acid (DTPA), and a combination thereof.

6. The method of claim 5, wherein the chelating agent further comprises a buffer selected from the group consisting of Tris buffer and phosphate buffer.

7. The method of claim 1, wherein the enzyme solution comprises trypsin, bromelain, pepsin, papain, or any combination thereof.

8. The method of claim 1, wherein the enzyme is used at a concentration in the range of from about 0.05% to about 10%.

9. The method of claim 1, wherein the method further comprises (d) treating the connective tissue with an alkaline solution.

10. The method of claim 9, wherein the alkaline solution comprises an alkali metal hydroxide, an alkaline earth metal hydroxide, an alkali metal carbonate, an alkaline earth metal carbonate, an alkali metal bicarbonate, alkaline earth metal bicarbonate or a mixture thereof.

11. The method of claim 9, wherein the treatment of the connective tissue with an alkaline solution precedes the treatment of the connective tissue with an enzyme.

12. The method of claim 9, wherein the method further comprises (e) precipitating collagen from the liquid mutable collagenous tissue by adding an inorganic precipitation agent.

13. The method of claim 12, wherein the inorganic precipitation agent is an inorganic salt.

14. The method of claim 12, wherein the inorganic precipitation agent is sodium chloride, potassium chloride or calcium chloride.

15. The method of claim 12, wherein the method further comprises separation of the precipitated collagen.

16. The method of claim 12, wherein the method further comprises (f) dialysis of the precipitated collagen from step (e).

17. The method of claim 16, wherein the method further comprises (g) freeze drying the dialyzed collagen of step (f) to produce a solid mutable collagenous tissue.

18. The method of claim 17, wherein the solid mutable collagenous tissue is a powder or a sheet.

19. The method of claim 17, wherein the solid mutable collagenous tissue is at least 90%, at least 95%, at least 96%, at least 97%, at least 98%, or at least 99% pure collagen type I.

20. The method of claim 17, wherein the solid mutable collagenous tissue is at least 95% pure collagen type I.

21-30. (canceled)

31. The method of claim 1, wherein the organic solvent is a non-reducing organic solvent.

32. The method of claim 1, wherein the enzyme solution comprises 0.05-1% enzyme, and treatment with the enzyme solution if for between about 48 to about 72 hours.

33. The method of claim 1, wherein the organic solvent is selected from ethyleneglycol, methanol, ethanol, isopropanol, pyridine, acetonitrile, nitromethane, dimethylsulfoxide, ethylacetate, hexylene glycol, 2,2-thiodiglycol, propylene glycol, dioxane, acetone, dicholoroethane, tetrahydrofuran, dicholoromethane, chloroform, diethylether, benzene, toluene, xylene, carbontetrachloride, cyclohexane, petroleum ether, hexane, pentane, and a combination thereof.

34. The method of claim 1, wherein the mutable collagenous tissue exhibits less than 2% impurities and degradation products that are substantially below 150 kDa.

35. The method of claim 1, wherein the method further comprises dialyzing the mutable collagenous tissue against deionized water to obtain a native mutable collagenous tissue solution.

36. The method of claim 1, wherein the chelating agent is present in a concentration of about 0.8 μM to about 1 μM.
